(19) United States
(12) Patent Application Publication
(10) Pub. No.: US 2009/0285919 A1
(43) Pub. Date: Nov. 19, 2009

(54) Rice Bran Extracts for Inflammation and Methods of Use Thereof

(76) Inventors: Randall S. Alberte, Estero, FL (US); William P. Roschek, JR., Naples, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/467,835

(22) Filed: May 18, 2009

Related U.S. Application Data
(60) Provisional application No. 61/054,151, filed on May 18, 2008.
(60) Provisional application No. 61/101,475, filed on Sep. 30, 2008.
(60) Provisional application No. 61/147,305, filed on Jan. 26, 2009.

(51) Int. Cl. A61K 36/899; A23L 1/28; A61P 29/00; A61P 25/00; A61P 35/00

(52) U.S. Cl. 424/750; 426/655

(57) ABSTRACT

The present invention relates in part to stabilized rice bran extracts enriched in compounds that have inhibitory activity against certain anti-inflammatory therapeutic endpoints, such as the COX-1, COX-2 and 5-LOX enzymes. Another aspect of the invention relates to pharmaceutical compositions comprising the extracts and to methods of treating inflammatory diseases comprising administering the aforementioned extracts.

### RELATED APPLICATIONS

This application claims the benefit of priority to U.S. Provisional Application Nos. 61/054,151, filed on May 18, 2008, 61/101,475, filed on Sep. 30, 2008, and 61/147,305, filed on Jan. 26, 2009, each of which is herein incorporated by reference in its entirety.

### BACKGROUND OF THE INVENTION

Rice (Oryza sativa) bran, comprising 10% of the total rice grain, is a by-product of rice milling industry with world production of about 50-60 million metric tons per year. Rice bran is an excellent source of lipids, especially unsaturated fatty acids. Rice bran oil contains an array of bio-active phytochemicals such as oryzanols, phytostetols, tocotrienols, flavonoids, vitamins, squalene, polycosanols, phytic acid, ferulic acid, inositol hexaphophate. Additional constituents of the bran include protein (11-15%), carbohydrates (34-62%), ash (7-10%), vitamins, minerals and crude fibers (7-11%) (M. C. Kik, 1956. Nutritive value of rice, nutrients in rice bran and rice polish and improvement of protein quality with amino acids, J. Agric. Food Chem. 4:170-172; C. A. Rohrer and T. J. Siebenmorgen, 2004. Nutraceutical concentrations within the bran of various Rrice kernel thickness fractions, Biosys. Eng. 88:453-460).

Rice bran oil contains 95.6% saponifiable lipids, including glycolipid and phospholipids; and 4.2% unsaponifiable lipids, including tocopherols, tocotrienols, γ-oryzanol, sterols and carotenoids. The saponifiable lipids are mainly triglycerides. However, these triglycerides are easily hydrolyzed by lipase to form fatty acids. γ-oryzanol content in the rice bran oil is approximately 0.98%-2.9%. The γ-oryzanol is a mixture of 10 ferulate esters of triterpene alcohol that have been characterized extensively. The γ-oryzanols protect rice bran oil from oxidation, inhibit peroxidation of lipids mediated by iron or UV irradiation, and has been shown to lower blood cholesterol and used to treat nerve imbalance (C. Aguilar-Garcia, G. Gavino, M. Baragano-Mosqueda, P. Hevia and V. C. Gavino, 2007. Correlation of tocopherol, tocotrienol, [gamma]-oryzanol and total polyphenol content in rice bran with different antioxidant capacity assays, Food Chem. 102:1228-1232; Ardiansyah, H. Shirakawa, T. Koseki, K. Ohinata, K. Hashizume and M. Komai, 2006. Rice bran fractions improve blood pressure, lipid profile, and glucose metabolism in stroke-prone spontaneously hypertensive rats, J. Agric. Food Chem. 54:1914-1920). The major components of γ-oryzanol in rice bran are cycloartenyl ferulate, 24-methylene cycloartanyl ferulate and campestanyl ferulate (S. Lilitchan, C. Tangprawat, K. Aryusuk, S. Krisnangkura, S. Chokmoh and K. Krisnangkura, 2008. Partial extraction method for the rapid analysis of total lipids and [gamma]-oryzanol contents in rice bran, Food Chem. 106:752-759).

Rice bran oil contains about 0. 1-0. 14% vitamin E. Vitamin E is a generic term for a group of four tocopherols (α-, β-, γ- and δ-) and four tocotrienols (α-, β-, γ- and δ-), of which α-tocopherol has the highest biological activity. All components of vitamin E have an amphiphilic structure with a hydrophilic (chromanol ring) and a hydrophobic dominant (isoprenoid side chain). A number of studies showed that vitamin E functions as a chain-breaking antioxidant that prevents the propagation of free radical reactions. Because of its radical scavenging antioxidant properties, vitamin E inhibits lipid peroxidation in vitro and in vivo. Tocotrienols also have antitumor action against breast cancers and possible beneficial effects on cardiovascular health, and they decrease serum total cholesterol and LDL cholesterol levels (Ardiansyah, H. Shirakawa, T. Koseki, K. Ohinata, K. Hashizume and M. Komai, 2006. Rice bran fractions improve blood pressure, lipid profile, and glucose metabolism in stroke-prone spontaneously hypertensive rats, J. Agric. Food Chem. 54:1914-1920; T. Akihisa, K. Yasukawa, M. Yamaura, M. Ukiya, Y. Kimura, N. Shimizu and K. Arai, 2000. Triterpene alcohol and sterol ferulates from rice bran and their anti-inflammatory effects, J. Agric. Food Chem. 48:2313-2319; A. Idouraine, M. J. Khan and C. W. Weber, 1996. In vitro binding capacity of wheat bran, rice bran, and oat fiber for Ca, Mg, Cu, and Zn alone and in different combinations, J. Agric. Food Chem. 44:2067-2072; E. H. Jung, S. Ran Kim, I. K. Hwang and T. Youl Ha, 2007. Hypoglycemic effects of a phenolic acid fraction of rice bran and ferulic acid in C57BL/KsJ-db/db mice, J. Agric. Food Chem. 55:9800-9804; R. Renuka Devi and C. Arumughan, 2007. Antiradical efficacy of phytochemical extracts from defatted rice bran, Food Chem. Toxicol. 45:2014-2021).

Various techniques used for extraction, isolation and purification of antioxidants from rice bran have been described in literature. (M. H. Chen and C. J. Bergman, 2005. A rapid procedure for analysing rice bran tocopherol, tocotrienol and [gamma]-oryzanol contents, Journal of Food Composition and Analysis. 18:319-331 )A rapid procedure for analyzing rice bran tocopherol, tocotrienol and oryzanol contents by using hexane, isopropanol and methanol as solvents has been developed (S. Lilitchan, C. Tangprawat, K. Aryusuk, S. Krisnangkura, S. Chokmoh and K. Krisnangkura, 2008. Partial extraction method for the rapid analysis of total lipids and [gamma]-oryzanol contents in rice bran, Food Chem. 106:752-759). It was found that the tocopherol, tocotrienol and oryzanol in fresh rice bran were 98.3 mg/g, 223.6 mg/g and 3.4-3.9 mg/g fresh bran weight. Renuka Devi et al. (R. Renuka Devi and C. Arumughan, 2007. Antiradical efficacy of phytochemical extracts from defatted rice bran, Food Chem. Toxicol. 45:2014-2021) provided (R. Renuka Devi and C. Arumughan, 2007. Phytochemical characterization of defatted rice bran and optimization of a process for their extraction and enrichment, Bioresource Technology. 98:3037-3043) a phytochemical characterization of defatted rice bran and optimization of a process for their extraction and enrichment. The yield of total phenols, oryzanols and ferulic acid with methanol were 0.22, 0.03 and 0.023%, respectively. Microwave assisted solvent extraction is a relatively new extraction method that has be used for oil extractions. More recently, supercritical carbon dioxide (SCCO₂) extractions have shown that the odor and the flavor of extracted oil are superior to that obtained by traditional solvent extraction. (C. Balachandran, P. N. Mayamol, S. Thomas, D. Sukumar, A. Sundaresan and C. Arumughan, 2008. An ecofriendly approach to process rice bran for high quality rice bran oil using supercritical carbon dioxide for nutraceutical applications, Bioresource Technology. 99:2905-2912) SCCO₂ extraction can overcome limitations of traditional techniques that affect extract quality. As a solvent, CO₂ is non-toxic and can be easily and completely removed from products; moreover, it is non-corrosive and non-flammable. In addition to the well characterized oil and fatty acid components of rice bran, rice bran is rich in phenolics, alkaloids, gingerols and terpenes.

The inflammatory cascades responsible for pain, join immobility and swelling in osteoarthritis (OA) and rheumatoid arthritis (RA) have been the subject of significant investigation (S. G. Trivedi, J. Newson, R. Rajakariar, T. S. Jacques, R. Hannon, Y. Kanaoka, N. Eguchi, P. Colville-Nash and D. W. Gilroy, 2006. Essential role for hematopoietic prostaglandin D2 synthase in the control of delayed type hypersensitivity, Proc. Natl. Acad. Sci. USA. 103:5179-5184; W. F. Kean and W. W. Buchanan, 2005. The use of NSAIDs in rheumatic disorders 2005: a global perspective, Inflammopharmacology. 13:343-370). Central to these pathways is arachidonic acid, which serves as the substrate for the COX-1 and COX-2 (Cyclooxygenase) enzymes as well as the family of lipoxygenases (W. F. Kean and W. W. Buchanan, 2005. The use of NSAIDs in rheumatic disorders 2005: a global perspective, Inflammopharmacology. 13:343-370; J. L. Masferrer, B. S. Zweifel, K. Seibert and P. Needleman, 1990. Selective regulation of cellular cyclooxygenase by dexamethasone and endotoxin in mice, J. Clin. Invest. 86:1375-1379; S. K. Kulkarni and V. P. Singh, 2008. Positioning dual inhibitors in the treatment of pain and inflammatory disorders, Inflammopharmacology. 16:1-15; J. N. Sharma and L. A. Mohammed, 2006. The role of leukotrienes in the pathophysiology of inflammatory disorders: is there a case for revisiting leukotrienes as therapeutic targets?, Inflammopharmacology. 14:10-16). COX as a target for OA was discovered in the early 1990's (J. L. Masferrer, B. S. Zweifel, K. Seibert and P. Needleman, 1990. Selective regulation of cellular cyclooxygenase by dexamethasone and endotoxin in mice, J. Clin. Invest. 86:1375-1379; W. L. Xie, J. G. Chipman, D. L. Robertson, R. L. Erikson and D. L. Simmons, 1991. Expression of a mitogen-responsive gene encoding prostaglandin synthase is regulated by mRNA splicing, Proc. Natl. Acad. Sci. USA. 88:2692-2696; D. A. Kubuju, B. S. Fletcher, B. C. Barnum, R. W. Lim and H. R. Herschman, 1991. TIS10, a phorbol ester tumor prompter-inducible mRNA from Swiss 3T3 cells, encodes a novel prostaglandin synthase/cyclooxygenase homologue, J. Biol. Chem. 266: 12866-12872). Investigators discovered a new gene product (COX) that was induced in vitro while others found that COX activity could be induced by cytokines such as interleukin-1 (IL-1) and inhibited by corticosteroids. Steroids inhibited the IL-1-induced COX activity but not basal COX activity. These observations led to the hypothesis that there were two COX isoenzymes, one of which was constitutively expressed and responsible for basal prostaglandin generation, while the other was induced by inflammatory stimuli such as IL-1 and suppressed by glucocorticoids. The COX-1 enzyme is constitutively expressed and is found in nearly all tissues and cells, while the inducible COX-2 enzyme is the major player in dramatically enhanced production of prostaglandins from arachidonic acid and their release at sites of inflammation.

COX-1 and COX-2 serve identical functions in catalyzing the conversion of arachidonic acid to prostanoids. The specific prostanoid(s) generated in any given cell is not determined by whether that cell expresses COX-1 or COX-2, but by which distal enzymes in the prostanoid synthetic pathways are expressed. Stimulated human synovial cells synthesize small amounts of PGE₂ and prostacyclin but not thromboxane (TxB₂), PGD, or PGF₂ₐ. Following exposure to IL-1, synovial cells make considerably more PGE₂ and prostacyclin, but they still do not synthesize PGD, TxB₂ or PGF₂ₐ (J. M. Bathon, F. H. Chilton, W. C. Hubbard, M. C. Towns, N. J. Solan and D. Proud, 1996. Mechanisms of prostanoid synthesis in human synovial cells: cytokine-peptide synergism, Inflammation. 20:537-554). The IL1-induced increase in PGE₂ and prostacyclin is mediated exclusively through COX-2 (L. J. Crofford, R. L. Wilder, A. P. Ristimaki, H. Sano, E. F. Remmers, H. R. Epps and T. Hla, 1994. Cyclooxygenase-1 and -2 expression in rheumatoid synovial tissues. Effects of interleukin-1 beta, phorbol ester, and corticosteroids, J. Clin. Invest. 93:1095-1101).

COX-1 is expressed in nearly all cells, indicating that at least low levels of prostanoids are important in serving critical physiological (homeostatic) functions in humans. COX-1-mediated production of prostaglandins in the stomach serves to protect the mucosa against the ulcerogenic effects of acid and other insults, and COX-1 mediated production of thromboxane in platelets promotes normal clotting. COX-2 levels, in contrast, are dramatically up-regulated in inflamed tissues. For example, COX-2 expression and concomitant PGE₂ production are greatly enhanced in rheumatoid synovium compared to the less inflamed osteoarthritic synovium, and in animal models of inflammatory arthritis (L. J. Crofford, R. L. Wilder, A. P. Ristimaki, H. Sano, E. F. Remmers, H. R. Epps and T. Hla, 1994. Cyclooxygenase-1 and -2 expression in rheumatoid synovial tissues. Effects of interleukin-1 beta, phorbol ester, and corticosteroids, J. Clin. Invest. 93:1095-1101; G. D. Anderson, S. D. Hauser, K. L. McGarity, M. E. Bremer, P. C. Isakson and S. A. Gregory, 1996. Selective inhibition of cyclooxygenase (COX)-2 reverses inflammation and expression of COX-2 and interleukin 6 in rat adjuvant arthritis, J. Clin. Invest. 97:2672-2679). This is clearly the result of excessive production of IL- 1, tumor necrosis factor and growth factors in the rheumatoid joint. Therefore, COX-2 selective inhibitors are highly desirable for both OA and RA, and are key to down-regulating the downstream production of pro-inflammatory prostaglandins and leukotrienes.

The generation of pro-inflammatory prostanoids is a hallmark of cyclooxygenase activity (W. F. Kean and W. W. Buchanan, 2005. The use of NSAIDs in rheumatic disorders 2005: a global perspective, Inflammopharmacology. 13:343-370). There are at least 4 major pathways to the production of prostaglandins, depending on the tissue. In OA and RA, the production of PGH₂ by COX-2 is converted to the pro-inflammatory prostanoid, PGE₂ by PGE₂ Synthase (F. Kojima, H. Naraba, S. Miyamoto, M. Beppu, H. Aoki and S. Kawai, 2004. Membrane-associated prostaglandin E synthase-1 is upregulated by proinflammatory cytokines in chondrocytes from patients with osteoarthritis, Arthritis Res. Ther. 6:R355-365; J. E. Jeffrey and R. M. Aspden, 2007. Cyclooxygenase inhibition lowers prostaglandin E2 release from articular cartilage and reduces apoptosis but not proteoglycan degradation following an impact load in vitro, Arthrit. Res. Ther. 9:R129). However, HPGD₂ Synthase, which plays a well established role in the inflammatory cascade associated with allergic rhinitis (R. L. Thurmond, E. W. Gelfand and P. J. Dunford, 2008. The role of histamine H1 and H4 receptors in allergic inflammation: the search for new antihistamines, Nat. Rev. Drug Discov. 7:41-53; S. T. Holgate and D. Broide, 2003. New targets for allergic rhinitis—a disease of civilization, Nat. Rev. Drug Discov. 2:902-914), has recently been shown to play an essential role in the control of hypersensitivity and persistent inflammation (S. G. Trivedi, J. Newson, R. Rajakariar, T. S. Jacques, R. Hannon, Y. Kanaoka, N. Eguchi, P. Colville-Nash and D. W. Gilroy, 2006. Essential role for hematopoietic prostaglandin D2 synthase in the control of delayed type hypersensitivity, Proc. Natl. Acad. Sci. USA. 103:5179-5184).The ant-inflammatory role of HPGD₂ outside of allergy is still somewhat unclear, but it is implicated as key to persistent inflammation.

The lipoxgenases also play a key pro-inflammatory role metabolizing arachidonic acid to leukotrienes. In particular 5- and 12-LOX are major players in this pathway (J. N. Sharma and L. A. Mohammed, 2006. The role of leukotrienes in the pathophysiology of inflammatory disorders: is there a case for revisiting leukotrienes as therapeutic targets?, Inflammopharmacology. 14:10-16; M. W. Whitehouse and K. D. Rainsford, 2006. Lipoxygenase inhibition: the neglected frontier for regulating chronic inflammation and pain, Inflammopharmacology. 14:99-102; L. Zhao, T. Grosser, S. Fries, L. Kadakia, H. Wang, J. Zhao and R. Falotico, 2006. Lipoxygenase and prostaglandin G/H synthase cascades in cardiovascular disease, Exp. Rev. Clin. Immunol. 2:649-658; J. Martel-Pelletier, D. Lajeunesse, P. Reboul and J. P. Pelletier, 2003. Therapeutic role of dual inhibitors of 5-LOX and COX, selective and non-selective non-steroidal anti-inflammatory drugs, Ann. Rheum. Dis. 62:501-509). Inhibition of COX-2 shunts arachidonic acid into the LOX pathways therefore a great deal of interest has been focused on co-inhibition of both COX and LOX pathways (W. F. Kean and W. W. Buchanan, 2005. The use of NSAIDs in rheumatic disorders 2005: a global perspective, Inflammopharmacology. 13:343-370; S. K. Kulkarni and V. P. Singh, 2008. Positioning dual inhibitors in the treatment of pain and inflammatory disorders, Inflammopharmacology. 16:1-15; J. N. Sharma and L. A. Mohammed, 2006. The role of leukotrienes in the pathophysiology of inflammatory disorders: is there a case for revisiting leukotrienes as therapeutic targets?, Inflammopharmacology. 14:10-16; M. W. Whitehouse and K. D. Rainsford, 2006. Lipoxygenase inhibition: the neglected frontier for regulating chronic inflammation and pain, Inflammopharmacology. 14:99-102; L. Zhao, T. Grosser, S. Fries, L. Kadakia, H. Wang, J. Zhao and R. Falotico, 2006. Lipoxygenase and prostaglandin G/H synthase cascades in cardiovascular disease, Exp. Rev. Clin. Immunol. 2:649-658; J. Martel-Pelletier, D. Lajeunesse, P. Reboul and J. P. Pelletier, 2003. Therapeutic role of dual inhibitors of 5-LOX and COX, selective and non-selective non-steroidal anti-inflammatory drugs, Ann. Rheum. Dis. 62:501-509). The LOX enzymes 5-, 12- and 15-LOX generate HpETE (hydroperoxy-eicosatrienoic acid—5, 12 or 15) end-products that serve as precursors for leukotrienes involved in pro- and anti-inflammatory pathways (H. Kuhn and V. B. O'Donnell, 2006. Inflammation and immune regulation by 12/15-lipoxygenases, Prog. Lipid Res. 45:334-356; H. Hikiji, T. Takato, T. Shimizu and S. Ishii, 2008. The roles of prostanoids, leukotrienes, and platelet-activating factor in bone metabolism and disease, Prog. Lipid Res. 47:107-126). In particular, 15-LOX has been implicated a variety of anti-inflammatory activities, particularly associated with vascular disease (H. Kuhn and V. B. O'Donnell, 2006. Inflammation and immune regulation by 12/15-lipoxygenases, Prog. Lipid Res. 45:334-356). In general 15-LOX enzymes are expressed by monocytes and macrophages after induction by T helper type 2 cytokines—IL-4 and IL-13.The products include the pro-inflammatory leukotrienes, as well as the anti-inflammatory lipoxins and hepoxilins (H. Kuhn and V. B. O'Donnell, 2006. Inflammation and immune regulation by 12/15-lipoxygenases, Prog. Lipid Res. 45:334-356). The activity of 5-LOX generates at least 4 specific leukotrienes, LTB₄, LTC₄, LTD₄ and LTE₄, and cytokines that contribute significantly to joint inflammation and bone resorption. Inhibition of 5-LOX is recognized a major therapeutic target for drug development for this diseases and related inflammatory diseases like asthma and certain vascular diseases (S. K. Kulkami and V. P. Singh, 2008. Positioning dual inhibitors in the treatment of pain and inflammatory disorders, Inflammopharmacology. 16:1-15).

Arthritis is an inflammation of the joints that can be chronic and is realized as joint swelling, immobility and pain. The disease, whether osteoarthritis, rheumatoid arthritis or gout, results from a dysregulation of pro-inflammatory cytokines (e.g., interleukins) and pro-inflammatory enzymes like COX and LOX that generate prostaglandins and leukotrienes, respectively. Fundamental to this pro-inflammatory process is the activation of nuclear transcription factor κB (NF-κB). As a consequence compounds that suppress the expression of TNF-α, COX and LOX, and their products, or NF-κB directly have significant potential for arthritis treatments. Current estimates suggest that by 2015 about 25% of the US population will suffer from various forms of arthritis, dramatically increasing the market for arthritis treatments from its current level of ca. $7.5 B to well over $15 B.

A majority of current drugs for arthritis are non-steroid anti-inflammatory agents (NSAIDs), and range from OTC products like Ibuprofen to prescription drugs like Celebrex. Most are non-selective COX-1 and COX-2 inhibitors (aspirin, ibuprofen, and naproxen) while others like Celebrex®, though not COX-2-specific, are highly selective for COX 2. COX-1 inhibitors, those drugs with high COX-1 to COX-2 selectivity, have significant side-effects due to the key anti-inflammatory role of COX-1 in prostaglandin production critical for protection of the gastric mucosa. Recently, it has been recognized that inhibition of COX-2 shunts arachidonic acid, the key substrate for inflammatory pathways, into leukotrienes primarily by up-regulation of 5-LOX (S. K. Kulkami and V. P. Singh, 2008. Positioning dual inhibitors in the treatment of pain and inflammatory disorders, Inflammopharmacology. 16:1-15; J. N. Sharma and L. A. Mohammed, 2006. The role of leukotrienes in the pathophysiology of inflammatory disorders: is there a case for revisiting leukotrienes as therapeutic targets?, Inflammopharmacology. 14:10-16; M. W. Whitehouse and K. D. Rainsford, 2006. Lipoxygenase inhibition: the neglected frontier for regulating chronic inflammation and pain, Inflammopharmacology. 14:99-102; L. Zhao, T. Grosser, S. Fries, L. Kadakia, H. Wang, J. Zhao and R. Falotico, 2006. Lipoxygenase and prostaglandin G/H synthase cascades in cardiovascular disease, Exp. Rev. Clin. Immunol 2:649-658; J. Martel-Pelletier, D. Lajeunesse, P. Reboul and J. P. Pelletier, 2003. Therapeutic role of dual inhibitors of 5-LOX and COX, selective and non-selective non-steroidal anti-inflammatory drugs, Ann. Rheum. Dis. 62:501-509; P. McPeak, R. Cheruvanky, C. R. S. V. and M. M., 2005. Methods for treating joint inflammation, pain, and loss of mobility. U.S. Pat. No. 6,902,739; Issued 7 Jul. 2005.). Therefore, significant effort has been directed towards the development of drugs or drug combinations that target both COX and 5-LOX (B. Naveau, 2005. Dual Inhibition of Cyclo-oxygenases and 5-Lipoxygenase: a Novel Therapeutic Approach to Inflammation?, Joint Bone Spine. 72:199-201). Licofelone is currently one of the most promising (S. K. Kulkarni and V. P. Singh, 2008. Positioning dual inhibitors in the treatment of pain and inflammatory disorders, Inflammopharmacology. 16:1-15; J. M. Alvaro-Gracia, 2004. Licofelone—clinical update on a novel LOX/COX inhibitor for the treatment of osteoarthritis, Rheumatol. 43 Suppl 1:21-25) and it has a favorable cardiovascular profile (G. Shoba, D. Joy, T. Joseph, M. Majeed, R. Rajendran and P. S. Srinivas, 1998. Influence of piperine on the pharmacokinetics of curcumin in animals and human volunteers, Planta Med. 64:353-356).

The 5-LOX enzyme is essential for transforming arachidonic acid into leukotrienes and has the ability to bind and possibly affect the function of a number of cellular proteins, including cytoskeletal proteins. Research into the 5-LOX pathway of the CNS indicates that 5-LOX may participate in a number of brain pathologies, including developmental neurometabolic diseases, strokes, seizures, Alzheimer's disease, age-associated neurodegeneration, prion disease, multiple sclerosis, and brain tumors. Physiologically, 5-LOX appears to be involved in neurogenesis. It is suggested that a new 5-LOX pharmacopoeia, which would be effective in the CNS would significantly advance research on the role of 5-LOX in the brain (H. Manev and T. Uz, 2002. 5-Lipoxygenase in the central nervous system: therapeutic implications Curr. Med. Chem. 1:115-121).

Several inflammatory processes play a critical role in brain aging and are associated with increased vulnerability to neurodegeneration. The COX-2 and 5-LOX enzymes are upregulated in the central nervous system during aging, and are associated with different aging-related brain pathologies. A COX-2 inhibitor has been shown to improve cognitive function in mice. In particular, COX-2 inhibition has been shown to significantly reverse the aging-induced retention deficit in mice. The COX and LOX inhibitors, and their combination, also have been shown to reverse the aging-induced motor dysfunction in the aged animals. On the basis of these observations, present findings indicate that the combination of COX and LOX inhibitors (dual inhibitors) may provide a new therapeutic innovation for the treatment of age-related brain disorders such as Alzheimer's disease and other motor dysfunctions with adequate gastrointestinal tolerability (M. Bishnoi, C. S. Patil, A. Kumar and S. K. Kulkarni, 2005. Protective effects of nimesulide (COX Inhibitor), AKBA (5-LOX Inhibitor), and their combination in aging-associated abnormalities in mice, Methods Find. Exp. Clin. Pharmacol. 27:465-470; D. Paris, T. Town, T. Parker, J. Humphrey and M. Mullan, 2000. A beta vasoactivity: an inflammatory reaction, Ann. N.Y. Acad. Sci. 903:97-109). Thus, both COX-1/COX-2 and 5-LOX activities increase with age and contribute to neuro-degeneration. Inhibition of these enzymes reduces this process.

Alzheimer's disease (AD) is the most common dementing illness of the elderly and is a mounting public health problem. Pharmacoepidemiological data, analytical data from human tissue and body fluids, and mechanistic data mostly from murine models all have implicated oxidation products of two fatty acids, arachidonic acid (AA) and docosahexaenoic acid (DHA), in the pathogenesis of neurodegeneration. Inhibition of COX-1, COX-2 and 5-LOX activity reduces neurotoxicity and neurodegeneration. These reactions that mediate AA metabolism are key to pathogenesis of dementias.

COX and LOX inhibitors also play a role in cancer pathogenesis. Previous studies indicate that the arachidonic acid-metabolizing enzymes COX-2 and 5-LOX are overexpressed during the process of colonic adenoma formation promoted by cigarette smoke. Pretreating colon cancer cells with cigarette smoke extract (CSE) promoted colon cancer growth in the nude mouse xenograft model. Inhibition of COX-2 or 5-LOX reduced the tumor size. In the group treated with a COX-2-inhibitor, the PGE₂ level decreased while the LTB₄ level increased. In contrast, in the 5-LOX-inhibitor treated group, the LTB₄ level was reduced and the PGE₂ level was unchanged. Notably, combined treatment with both COX-2 and 5-LOX inhibitors further inhibited the tumor growth promoted by CSE over treatment with either COX-2 inhibitor or 5-LOX inhibitor alone. In an in vitro study, the action of CSE on colon cancer cells was mediated by 5-LOX DNA demethylation. These results indicate that inhibition of COX-2 may lead to a shunt of arachidonic acid metabolism towards the leukotriene pathway during colonic tumorigenesis promoted by CSE. Suppression of 5-LOX did not induce such a shunt and produced a better response. Therefore, 5-LOX inhibitor is more effective than COX-2 inhibition, and inhibition of both COX-2 and 5-LOX may present a superior anticancer profile in cigarette smokers (Y. N. Ye, W. K. Wu, V. Y. Shin, I. C. Bruce, B. C. Wong and C. H. Cho, 2005. Dual inhibition of 5-LOX and COX-2 suppresses colon cancer formation promoted by cigarette smoke, Carcinogenesis. 26:827-834).

Selective inhibition of eicosanoid synthesis seems to decrease carcinogenesis, however, the effect on liver metastasis in pancreatic cancer is still unknown. Combined therapy (Celebrex® [COX-2 inhibitor]+Zyflo [5-LOX inhibitor]) significantly decreased incidence, number and size of liver metastases. Furthermore extra- and intra-metastatic concentration of PGE₂ was reduced by this treatment in hepatic tissue. COX-2-inhibition alone (Celebrex®) decreased intrametastatic hepatic PGF_{1α} and PGE₂ concentration while PGF_{1α} concentration was reduced in non-metastatic liver (nml). Moreover 5-LOX inhibition alone using Zyflo decreased intrametastatic PGE₂ concentration as well as PGF_{1α} and PGE₂ in nml. In pancreatic carcinomas highest LT-concentration was found after combined treatment and this therapy group was the only one revealing a significantly higher amount of LTs in carcinomas compared to tumor-free tissue. Hepatic LT-concentration was significantly lower in the control groups than in nml of the tumor groups. Thus, combination of COX-2 inhibition and 5-LOX inhibition might be a suitable adjuvant therapy to prevent liver metastasis in human ductal pancreatic adenocarcinoma (J. I. Gregor, M. Kilian, I. Heukamp, C. Kiewert, G. Kristiansen, I. Schimke, M. K. Walz, C. A. Jacobi and F. A. Wenger, 2005. Effects of selective COX-2 and 5-LOX inhibition on prostaglandin and leukotriene synthesis in ductal pancreatic cancer in Syrian hamster, Prostag. Leukotr. Ess. Fatty Acids. 73:89-97).

Emerging reports now indicate alterations of arachidonic acid metabolism with carcinogenesis and many COX and LOX inhibitors (used for the treatment of inflammatory diseases) are being investigated as potential anticancer drugs. Results from clinical trials seem to be encouraging but a better knowledge of the dynamic balance that shifts toward lipoxygenases (and different LOX isoforms) and COX-2 are essential to progress in the design of new drugs specially directed to chemoprevention or chemotherapy of human cancers. On the basis of these results, it was useful to study the advantages of COX inhibitor and LOX inhibitor combinations and a next step will be the conception of dual inhibitors able to induce the anticarcinogenic and/or to inhibit the procarcinogenic enzymes responsible for polyunsaturated fatty acid metabolism (L. Goossens, N. Pommery and J. P. Henichart, 2007. COX-2/5-LOX dual acting anti-inflammatory drugs in cancer chemotherapy, Curr. Top. Med. Chem. 7:283-296).

The effects of 5-LOX or 12-LOX inhibitors on human breast cancer cell proliferation and apoptosis have been studied. The LOX inhibitors, NDGA, Rev-5901, and baicalein all inhibited proliferation and induced apoptosis in MCF-7 (ER+) and MDA-MB-23 1 (ER−) breast cancer cells in vitro. In contrast, the LOX products, 5-HETE and 12-HETE had mitogenic effects, stimulating the proliferation of both cell lines. These inhibitors also induced cytochrome c release, caspase-9 activation, as well as downstream caspase-3 and caspase-7 activation, and PARP cleavage. LOX inhibition also reduced the levels of anti-apoptotic proteins Bc1-2 and Mcl-1 and increased the levels of the pro-apoptotic protein bax. Thus, blockade of both 5-LOX and 12-LOX pathways induces apoptosis in breast cancer cells through the cytochrome c release and caspase-9 activation, with changes in the levels of Bc1-2 family proteins (W. G. Tong, X. Z. Ding and T. E. Adrian, 2002. The mechanisms of lipoxygenase inhibitor-induced apoptosis in human breast cancer cells, Biochem. Biophys. Res. Commun. 296:942-948).

COX-2 inhibitors are efficacious as the non-selective NSAIDs for the treatment of postoperative pain, but have the advantages of a better gastrointestinal side-effect profile as well as a lack of antiplatelet effects. There have been recent concerns regarding the cardiovascular side effects of COX-2 inhibitors. Nonetheless, they remain a valuable option for postoperative pain management (N. M. Gajraj, 2007. COX-2 inhibitors celecoxib and parecoxib: valuable options for postoperative pain management, Curr. Top. Med. Chem. 7:235-249).

Dual 5-LOX/COX-2 inhibitors are potential new drugs to treat inflammation. They act by blocking the formation of both prostaglandins and leukotrienes but do not affect lipoxin formation. Such combined inhibition avoids some of the disadvantages of selective COX-2 inhibitors, spares the gastrointestinal mucosa, and are highly effective for pain mitigation (J. Martel-Pelletier, D. Lajeunesse, P. Reboul and J. P. Pelletier, 2003. Therapeutic role of dual inhibitors of 5-LOX and COX, selective and non-selective non-steroidal anti-inflammatory drugs, Ann. Rheum. Dis. 62:501-509).

NSAID management of the inflammatory process has focused on reducing the production of inflammatory prostaglandins by inhibiting the COX enzymes. However, blocking COX also reduces gastroprotective prostaglandins, causing the well-known gastrointestinal side effects. Furthermore, a shunting of arachidonic acid to the 5-LOX pathway may also occur, causing an increase in leukotrienes and further GI damage. Pharmacodynamic studies determined that ML3000, a dual inhibitor of COX and 5-LOX, with analgesic, anti-inflammatory, antipyretic, antiplatelet, and anti-bronchoconstrictive activity, had minimal gastrointestinal side effects. Clinical studies show efficacy in osteoarthritis and excellent gastrointestinal safety (S. Laufer, 2001. Discovery and development of ML3000, Inflammopharmacology. 9:101-112).

Botanicals from both Traditional Chinese Medicine (TCM) and Ayurveda Medicine, the traditional medicine of India, have a long use history for arthritis and inflammatory diseases (D. Khanna, G. Sethi, K. S. Ahn, M. K. Pandey, A. B. Kunnumakkara, B. Sung, A. Aggarwal and B. B. Aggarwal, 2007. Natural products as a gold mine for arthritis treatment, Curr. Opin. Pharm. 7:344-351). Botanicals have certain benefits for treating diseases like arthritis that involve multiple cellular/molecular targets and manifest themselves in several different ways because of the potential synergies that can accrue from the chemical diversity present.

Though there is significant historical use of a broad variety of botanicals for arthritis (D. Khanna, G. Sethi, K. S. Ahn, M. K. Pandey, A. B. Kunnumakkara, B. Sung, A. Aggarwal and B. B. Aggarwal, 2007. Natural products as a gold mine for arthritis treatment, Curr. Opin. Pharm. 7:344-351), only about 18 bioactives from about the same number of botanicals have been identified to date that have significant COX, LOX and related targets for anti-arthritis activities (MMP-9, TNFα, ICAM-1). It would therefore be desirable to provide a stabilized rice bran extract having high concentrations of compounds with high COX-1, COX-2, and 5-LOX inhibiting activities

Disclosed herein are optimized extracts from stabilized rice bran possessing very high anti-inflammatory activities targeting the COX-1, COX-2, and 5-LOX enzymes which are major mediators of inflammation, pain and joint immobility in arthritis. These extracts hold great promise for natural treatments for arthritis including joint pain and immobility and other inflammatory disorders. These extracts are safe and efficacious, and can be provided as dietary supplements, added to multiple vitamins, and incorporated into foods to create functional foods.

### SUMMARY OF THE INVENTION

The present invention relates in part to stabilized rice bran (SRB) extracts of the present invention that are useful for treating or preventing inflammation and arthritis, and/or pain associated with these conditions as well as neurodegenerative disorders effected by COX and LOX enzymes. As disclosed herein, preferred extracts are enriched in a range of bioactives that address several important and key inflammation and arthritis therapeutic endpoints.

One aspect of the invention relates to extracts of stabilized rice bran comprising an enriched amount of certain compounds having anti-inflammatory activity. The compounds have inhibitory activity against COX-1, COX-2, 5-LOX, or combinations thereof. Compounds include valeric/methylbutyric acid, norcamphor/heptadienal, conyrin, 6-methyl-5-hepten-2-one, ocimene/camphene/adamantane, histidinol, lysine, carvacrol/thymol/cymenol, 2,6-tropanediol, tryptamine, 2,4-hexanienoic acid isobutylamide, nonanedioic acid anhydride, acetylaburnine, nonanedioic acid diamide, epiloliolide, curcumene, farnesatrienetriol, farnesylacetone, octadecatrienol, hydroxyoctadecatrienoic acid, epoxyhydroxyoctadecanoic acid, and 12-shogoal. The SRB extract may contain any combination of the aforementioned compounds, or it may even contain all of the aforementioned compositions.

In some aspects of the invention, pharmaceutical formulations comprising any of the aforementioned and at least one pharmaceutically acceptable carrier are provided.

The aforementioned extracts or pharmaceutical compositions may be administered to a subject in need thereof for treatment or prevention of a variety of disease and conditions. Additionally, the compositions may be administered for the treatment or relief of the symptoms of a variety of conditions. When the symptoms of a disease or condition are treated or prevented, the underlying disease or condition may or may not be treated or prevented, depending on the particular disease or condition.

Further features and advantages of the disclosed extracts will become apparent from the description, drawings and claims that follow.

### BRIEF DESCRIPTION OF THE DRAWINGS

FIG. 1 depicts a diagram of the role of arachidonic acid in the proinflammation pathways involving COX-1, COX-2 and LOX.

FIG. 2 depicts a DART TOF-MS spectrum of SRB Extract 1 (extracted at 40° C., 80% ethanol), with the X-axis showing the mass distribution (100-800 m/z [M+H+]) and the y-axis showing the relative abundances of each chemical species of the detected.

FIG. 3 depicts a DART TOF-MS spectrum of SRB Extract 2 (obtained by Super critical CO₂ extraction at 40° C., 300 bar), with the X-axis showing the mass distribution (100-800 m/z [M+H+]) and the y-axis showing the relative abundances of each chemical species of the detected.

FIG. 4 depicts a DART TOF-MS spectrum of SRB extract 3 (mixture of SRB Extract 1 and SRB Extract 2 in a by weight ratio of 1:7), with the X-axis showing the mass distribution (100-800 m/z [M+H+]) and the y-axis showing the relative abundances of each chemical species of the detected.

FIG. 5 depicts pharmacokinetic profile of key bioactives of SRB Extract 3 that are bioavailable in serum as determine by DART TOF-MS.

FIG. 6 depicts the pharmacokinetic profile of key bioactives of SRB Extract 3 in urine as determined by DART TOF-MS.

### DETAILED DESCRIPTION OF THE INVENTION

### Definitions

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “extract” refers to a product prepared by extraction. The extract may be in the form of a solution in a solvent, or the extract may be a concentrate or essence which is free of, or substantially free of solvent. The term extract may be a single extract obtained from a particular extraction step or series of extraction steps, or the extract also may be a combination of extracts obtained from separate extraction steps. For example, extract “a” may be obtained by extracting SRB with alcohol in water, while extract “b” may be obtained by super critical carbon dioxide extraction of SRB. Extracts a and b may then be combined to form extract “c”. Such combined extracts are thus also encompassed by the term “extract.”

As used herein, the term “fraction” means the extract comprising a specific group of chemical compounds characterized by certain physical, chemical properties or physical or chemical properties.

As used herein, the term “profile” refers to the ratios by percent mass weight of the chemical compounds within an extraction fraction or to the ratios of the percent mass weight of each of the chemical constituents in a final SRB extract.

As used herein, the term “purified” fraction or composition means a fraction or composition comprising a specific group of compounds characterized by certain physical-chemical properties or physical or chemical properties that are concentrated to greater than 50% of the fraction's or composition's chemical constituents. In other words, a purified fraction or composition comprises less than 50% chemical constituent compounds that are not characterized by certain desired physical-chemical properties or physical or chemical properties that define the fraction or composition.

The term “synergistic” is art recognized and refers to two or more components working together so that the total effect is greater than the sum of the components.

The term “treating” is art-recognized and refers to curing as well as ameliorating at least one symptom of any condition or disorder.

A “patient,” “subject” or “host” to be treated by the subject method may be a primate (e.g. human), bovine, ovine, equine, porcine, rodent, feline, or canine.

The term “pharmaceutically acceptable salts” is art-recognized and refers to the relatively non-toxic, inorganic and organic acid addition salts of compounds, including, for example, those contained in compositions of the present invention. Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically-acceptable metal cation or with ammonia or an organic primary, secondary, or tertiary amine. Pharmaceutically-acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The term “effective amount” as used herein refers to the amount necessary to elicit the desired biological response. As will be appreciated by those of ordinary skill in this art, the effective amount of a drug may vary depending on such factors as the desired biological endpoint, the drug to be delivered, the composition of the encapsulating matrix, the target tissue, etc.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of an infection includes, for example, reducing the number of diagnoses of the infection in a treated population versus an untreated control population, and/or delaying the onset of symptoms of the infection in a treated population versus an untreated control population.

As used herein, the term “inhibitor” refers to molecules that bind to enzymes and decrease their activity. The binding of an inhibitor can stop a substrate from entering the enzyme's active site and/or hinder the enzyme from catalyzing its reaction. Inhibitor binding is either reversible or irreversible. Irreversible inhibitors usually react with the enzyme and change it chemically. These inhibitors modify key amino acid residues needed for enzymatic activity. Reversible inhibitors bind non-covalently and different types of inhibition are produced depending on whether these inhibitors bind the enzyme, the enzyme-substrate complex, or both.

As used herein, the term “inflammation” refers to the complex biological response of vascular tissues to harmful stimuli, such as pathogens, damaged cells, or irritants. It is a protective attempt by the organism to remove the injurious stimuli as well as initiate the healing process for the tissue. Inflammation is not a synonym for infection. Even in cases where inflammation is caused by infection, the two are not synonymous: infection is caused by an exogenous pathogen, while inflammation is the response of the organism to the pathogen.

As used here, the term “COX” refers to Cyclooxygenase (a.k.a. prostaglandin synthase, prostaglandin synthetase), an enzyme (EC 1.14.99.1) responsible for formation of important biological mediators called prostanoids (e.g., prostaglandins, prostacyclin and thromboxane). Inhibition of COX can provide relief from the symptoms of inflammation and pain. Non-steroidal anti-inflammatory drugs, such as the well-known aspirin and ibuprofen, act by inhibiting this enzyme.

As used herein, the term “Lipoxygenases” (LOX) refers to a family of iron-containing enzymes that catalyse the dioxygenation of polyunsaturated fatty acids in lipids containing a cis, cis-1,4-pentadiene structure.

As used herein, the term “Prostanoid” refers to a subclass of eicosanoids consisting of: the prostaglandins (mediators of inflammatory and anaphylactic reactions), the thromboxanes (mediators of vasoconstriction) and the prostacyclins (active in the resolution phase of inflammation).

As used herein, the term “Eicosanoids” refers to signaling molecules made by oxygenation of twenty-carbon essential fatty acids. There are four families of eicosanoids—the prostaglandins, prostacyclins, the thromboxanes and the leukotrienes.

As used herein, the term “Leukotrienes” refers to naturally produced eicosanoid lipid mediators responsible for the effects an inflammatory response. Leukotrienes use both autocrine and paracrine signalling to regulate the body's response. Leukotrienes are produced in the body from arachidonic acid by the enzyme 5-lipoxygenase.

As used herein, the term “Autocrine” refers to a form of signaling in which a cell secretes a hormone, or chemical messenger (called the autocrine agent) that binds to autocrine receptors on the same cell, leading to changes in the cell.

As used herein the term “Paracrine” refers to a form of cell signaling in which the target cell is different, but near (“para”=near) the signal-releasing cell.

As used herein the term “Arachidonic acid” (AA, sometimes ARA) refers to an omega-6 fatty acid 20:4(ω-6).

As used here, the term “Prostaglandin D2 Synthase”, or “HPGDS” refers to a glutathione-independent prostaglandin D synthase that catalyzes the conversion of prostaglandin H2 (PGH2) to prostaglandin D2 (PGD2). PGD2 functions as a neuromodulator as well as a trophic factor in the central nervous system. PGD2 has been shown to function as a mast cell mediator in triggering asthma and vasodilation.

As used herein, the term “Tau” refers to a class of microtubule-associated proteins that are abundant in neurons in the central nervous system. Tau proteins interact with tubulin to stabilize microtubules and promote tubulin assembly into microtubules. Tau has two ways of controlling microtubule stability: isoforms and phosphorylation. Six tau isoforms exist in brain tissue, and they are distinguished by their number of binding domains.

As used herein, the term “Tau phosphorylation” or “Tau hyper-phosphorylation” refers phosphorylation of tau via a host of kinases. For example, when PKN, a serine/threonine kinase is activated, it phosphorylates tau, resulting in disruption of microtubule organization. Hyper-phosphorylation of the tau protein (tau inclusions), however, can result in the self-assembly of tangles of paired helical filaments and straight filaments, which are involved in the pathogenesis of Alzheimer's disease and other tau pathologies.

As used herein, the term “AD” refers to Alzheimer's Disease which is a degenerative and terminal disease that is the most common form of dementia. AD has been identified as a protein misfolding disease due to the accumulation of abnormally folded amyloid beta protein in the brains of AD patients.

Extracts

One aspect of the invention relates to extracts of stabilized rice bran comprising an enriched amount of certain compounds having anti-inflammatory activity. The compounds have inflammatory activity against COX-1, COX-2, 5-LOX, or combinations thereof.

As described in further detail below, the compounds in the SRB extracts are identified by mass spectrometry. In certain instances, the precise identity of the structure could be one of two or three different chemicals. These instances are represented by a slash “/” between the chemical names, e.g., “norcamphor/heptadienal”. When represented as such, the SRB extract is intended to encompass one or all of the listed compounds.

In one aspect of the invention, the SRB extracts comprise at least one compound selected from the group consisting of valeric/methylbutyric acid, norcamphor/heptadienal, conyrin, 6-methyl-5-hepten-2-one, ocimene/camphene/adamantane, histidinol, lysine, carvacrol/thymol/cymenol, 2,6-tropanediol, tryptamine, 2,4-hexanienoic acid isobutylamide, nonanedioic acid anhydride, acetylaburnine, nonanedioic acid diamide, epiloliolide, curcumene, farnesatrienetriol, farnesylacetone, octadecatrienol, octadecatrienoic acid, hydroxyoctadecatrienoic acid, hydroxyoctadecenoic acid epoxyhydroxyoctadecanoic acid, and 12-shogaol. The SRB extracts comprise at least one of the aforementioned compounds, and in many embodiments, the extracts comprise more than one or several of the aforementioned compounds. The SRB extract may contain any combination of the aforementioned compounds, or it may even contain all of the aforementioned compositions. Examples of certain combinations of the aforementioned compounds are described further below.

In some embodiments, the SRB extract comprises at least one compound selected from the group consisting of 0.01 to 10% by weight valeric/methylbutyric acid, 0.01 to 10% by weight of norcamphor/heptadienal, 0.01 to 10% by weight conyrin, 0.05 to 10% by weight ocimene/camphene/adamantane, 0.01 to 10% by weight lysine, 0.05 to 10% by weight carvacrol/thymol/cymenol, 0.01 to 10% by weight nonanedioic acid anhydride, 0.05 to 10% by weight epiloliode, and 0.01 to 10% by weight of 12-shogoal.

In other embodiments, the SRB extract comprises at least one compound selected from the group consisting of 0.01 to 2% by weight valeric/methylbutyric acid, 0.05 to 3% by weight of norcamphor/heptadienal, 0.01 to 2% by weight conyrin, 0.05 to 3% by weight ocimene/camphene/adamantane, 0.05 to 3% by weight lysine, 0.1 to 5% by weight carvacrol/thymol/cymenol, 0.01 to 2% by weight nonanedioic acid anhydride, 0.1 to 5% by weight epiloliolide, and 0.01 to 2% by weight of 12-shogaol.

In other embodiments, the SRB extract comprises at least one compound selected from the group consisting of 5 to 300 μg valeric/methylbutyric acid, 50 to 500 μg norcamphor/heptadienal, 5 to 300 μg conyrin, 100 to 1,000 μg ocimene/camphene/adamantane, 50 to 500 μg lysine, 100 to 1,000 μg carvacrol/thymol/cymenol, 10 to 500 μg nonanedioic acid anhydride, 100 to 1000 μg epiloliolide, and 5 to 500 μg 12-shogoal, per 100 mg of the extract.

In other embodiments, the SRB extract comprises carvacrol/thymol/cymenol, 5 to 30% valeric/methylbutyric acid by weight of the carvacrol/thymol/cymenol, 10 to 50% norcamphor/heptadienal by weight of the carvacrol/thymol/cymenol, 1 to 20% conyrin by weight of the carvacrol/thymol/cymenol, 75 to 125% ocimene/camphene/adamantine by weight of the carvacrol/thymol/cymenol, 10 to 50% lysine by weight of the carvacrol/thymol/cymenol, 5 to 50% nonanedioic acid anhydride, 75 to 125% epiloliolide by weight of the carvacrol/thymol/cymenol, and 5 to 50% 12-shogoal by weight of the carvacrol/thymol/cymenol.

In some embodiments, the extract comprises at least one compound selected from the group consisting of 0.05 to 10% 6-methyl-5-hepten-2-one, 0. 1 to 10% histidinol, 0.05 to 10% 2,6-tropanediol, 0.05 to 10% tryptamine, 0.01 to 5% 2,4-hexanienoic acid isobutylamide, 0.01 to 5% acetylaburnine, 0.01 to 5% nonanedioic acid diamide, 0.05 to 10% curcumene, 0.05 to 10% farnesatrienetriol, 0. 1 to 20% farnesylacetone, 0.1 to 10% octadecatrienol, 0.5 to 20% octadecatrienoic acid, 0.1 to 10% hydroxyoctadecatrienoic acid, 0.1 to 20% hydroxyoctadecenoic acid, and 0.1 to 10% epoxyhydroxyoctadecanoic acid.

In other embodiments, the extract comprises at least one compound selected from the group consisting of 0.05 to 2% 6-methyl-5-hepten-2-one, 0.1 to 2% histidinol, 0.05 to 2% 2,6-tropanediol, 0.05 to 2% tryptamine, 0.01 to 1% 2,4-hexanienoic acid isobutylamide, 0.01 to 3% acetylaburnine, 0.01 to 2% nonanedioic acid diamide, 0.05 to 2% curcumene, 0.1 to 2% farnesatrientriol, 0.5 to 5% farnesylacetone, 0.1 to 2% octadecatrienol, 1 to 10% octadecatrienoic acid, 0.1 to 2% hydroxyoctadecatrienoic acid, 0.5 to 5% hydroxyoctadecenoic acid, and 0.1 to 2% epoxyhydroxyoctadecanoic acid.

In other embodiments, the extract comprises 25 to 1000 μg 6-methyl-5-hepten-2-one, 100 to 2000 μg histidinol, 25 to 500 μg 2,6-tropanediol, 10 to 500 μg tryptamine, 5 to 100 μg 2,4-hexanienoic acid isobutylamide, 10 to 500 μg acetylaburnine, 10 to 500 μg nonanedioic acid diamide, 25 to 500 μg curcumene, 50 to 1000 farnesatrientriol, 500 to 5000 μg farnesylacetone, 100 to 2000 μg octadecatrienol, 500 to 10,000 μg octadecatrienoic acid, 100 to 2000 μg hydroxyoctadecatrienoic acid, 100 to 2000 μg hydroxyoctadecenoic acid, and 50 to 2000 μg epoxyhydroxyoctadecanoic acid.

In some embodiments, the extract comprises octadecatrienoic acid, 1 to 20% 6-methyl-5-hepten-2-one by weight of the octadecatrienoic acid, 5 to 50% histidinol by weight of the octadecatrienoic acid, 1 to 20% 2,6-tropanediol by weight of the octadecatrienoic acid, 0.5 to 15% tryptamine by weight of the octadecatrienoic acid, 0.1 to 5% 2,4-hexanienoic acid isobutylamide by weight of the octadecatrienoic acid, 0.5 to 10% acetylaburnine by weight of the octadecatrienoic acid, 0.5 to 10% nonanedioic acid diamide by weight of the octadecatrienoic acid, 1 to 15% curcumene by weight of the octadecatrienoic acid, 1 to 25% farnesatrientriol by weight of the octadecatrienoic acid, 10 to 75% farnesylacetone by weight of the octadecatrienoic acid, 5 to 50% octadecatrienol by weight of the octadecatrienoic acid, 5 to 50% hydroxyoctadecatrienoic acid by weight of the octadecatrienoic acid, 5 to 50% hydroxyoctadecenoic acid by weight of the octadecatrienoic acid, and 1 to 20% epoxyhydroxyoctadecanoic acid by weight of the octadecatrienoic acid.

In another embodiment, the stabilized rice bran extract comprises at least one compound selected from the group consisting of 0.001 to 5% norcamphor/heptadienal, 0.05 to 5% 6-methyl-5-hepten-2-one, 0.001 to 5% ocimene/camphene/adamantane, 0.05 to 5% histidinol, 0.001 to 5% lysine, 0.001 to 5% tryptamine, 0.05 to 5% nonanedioic acid anhydride, 0.05 to 5% nonanedioic acid diamide, 0.05 to 5% epiloliolide, 0.05 to 5% farnesatrientriol, 0.1 to 10% farnesylacetone, 0.1 to 10% octadecatrienol, 1 to 10% octadecatrienoic acid, 0.1 to 10% hydroxyoctadecatrienoic acid, 0.1 to 5% hydroxyoctadecenoic acid, 0.1 to 5% epoxyhydroxyoctadecanoic acid, and 0.1 to 5% 12-shogaol.

In another embodiment, the stabilized rice bran extract comprises at least one compound selected from the group consisting of 0.001 to 1% norcamphor/heptadienal, 0.05 to 1% 6-methyl-5-hepten-2-one, 0.001 to 1% ocimene/camphene/adamantane, 0.05 to 1% histidinol, 0.001 to 1% lysine, 0.001 to 1% tryptamine, 0.05 to 1% nonanedioic acid anhydride, 0.05 to 1% nonanedioic acid diamide, 0.05 to 1% epiloliolide, 0.05 to 1% farnesatrientriol, 0.5 to 2% farnesylacetone, 0.1 to 1% octadecatrienol, 1 to 5% octadecatrienoic acid, 0.5 to 2% hydroxyoctadecatrienoic acid, 0.1 to 1% hydroxyoctadecenoic acid, 0.1 to 1% epoxyhydroxyoctadecanoic acid, and 0.1 to 1.5% 12-shogaol.

In another embodiment, the stabilized rice bran extract comprises at least one compound selected from the group consisting of 5 to 100 μg norcamphor/heptadienal, 10 to 500 μg 6-methyl-5-hepten-2-one, 5 to 100 μg ocimene/camphene/adamantane, 10 to 500 μg histidinol, 5 to 100 μg lysine, 5 to 100 μg tryptamine, 100 to 500 μg nonanedioic acid anhydride, 10 to 100 μg nonanedioic acid diamide, 50 to 1000 μg epiloliolide, 10 to 1000 μg farnesatrienetriol, 100 to 5000 μg famesylacetone, 50 to 2500 μg octadecatrienol, 500 to 10000 μg octadecatrienoic acid, 100 to 5000 μg hydroxyoctadecatrienoic acid, 100 to 2500 μg hydroxyoctadecenoic acid, 50 to 1500 μg epoxyhydroxyoctadecanoic acid, and 100 to 2500 μg 12-shogoal, per 100 mg of the extract.

In another embodiment, the stabilized rice bran extract comprises octadecatrienoic acid, 0.1 to 5% norcamphor/heptadienal by weight of the octadecatrienoic acid, 0.5 to 10% 6-methyl-5-hepten-2-one by weight of the octadecatrienoic acid, 0.1 to 5% ocimene/camphene/adamantine by weight of the octadecatrienoic acid, 0.5 to 10% histidinol by weight of the octadecatrienoic acid, 0.1 to 5% lysine by weight of the octadecatrienoic acid, 0.1 to% tryptamine by weight of the octadecatrienoic acid, 0.1 to 10 % nonanedioic acid anhydride by weight of the octadecatrienoic acid, 0.1 to 10% nonanedioic acid diamide by weight of the octadecatrienoic acid, 1 to 20% epiloliolide by weight of the octadecatrienoic acid, 1 to 20% famesatrientriol by weight of the octadecatrienoic acid, 5 to 75% famesylacetone by weight of the octadecatrienoic acid, 5 to 50% octadecatrienol by weight of the octadecatrienoic acid, 5 to 75% hydroxyoctadecatrienoic acid by weight of the octadecatrienoic acid, 5 to 50% hydroxyoctadecenoic acid by weight of the octadecatrienoic acid, 5 to 50% epoxyhydroxyoctadecanoic acid by weight of the octadecatrienoic acid, and 5 to 50% 12-shogaol by weight of the octadecatrienoic acid.

In some embodiments, the SRB extract is prepared by a process comprising the following steps:

a) providing a stabilized rice bran feedstock, and

b) extracting the feedstock.

In some embodiments, the extracting step is an aqueous, alcoholic, or aqueous-alcoholic extract. For example, the extraction may be 100% water, or 100% alcohol, or any combination of water and alcohol, such as 10-95% alcohol, or 20-80% alcohol. In certain embodiments, the extract is 20, 40, 60, or 80% alcohol, while in other embodiments, the extraction is 30 to 50% alcohol. In some embodiments, the alcohol is ethanol. For example, the extraction may be about 40% ethanol at about 40 degrees Celsius. In other embodiments, the extraction may be by super critical CO₂ extraction, for example, SS CO₂ extraction at about 20-100° C., at a pressure of 200 to 600 bar. In certain embodiments, the extraction is at about 40 degrees Celsius and about 300 bar. In yet another embodiment, an extract is prepared by combining an extract prepared by aqueous or alcoholic extraction and an extract prepared by SSCO₂ extraction.

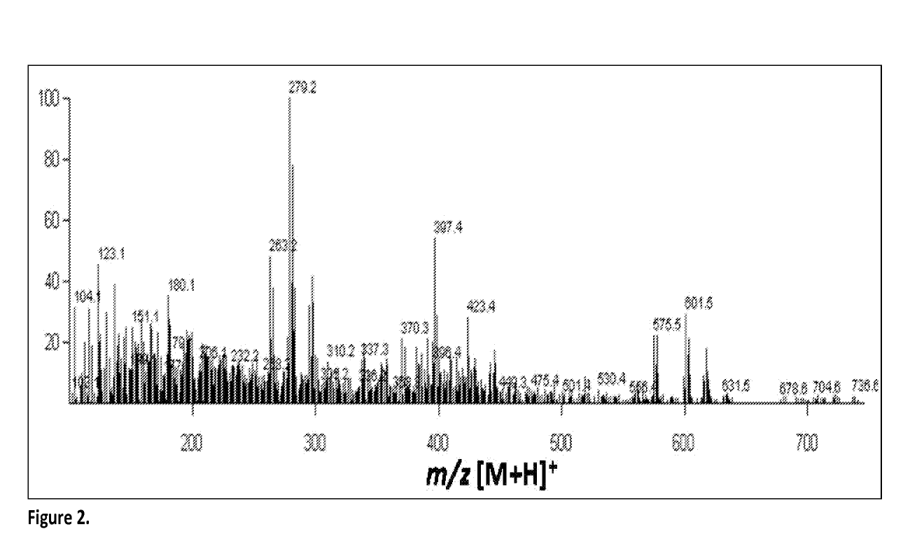

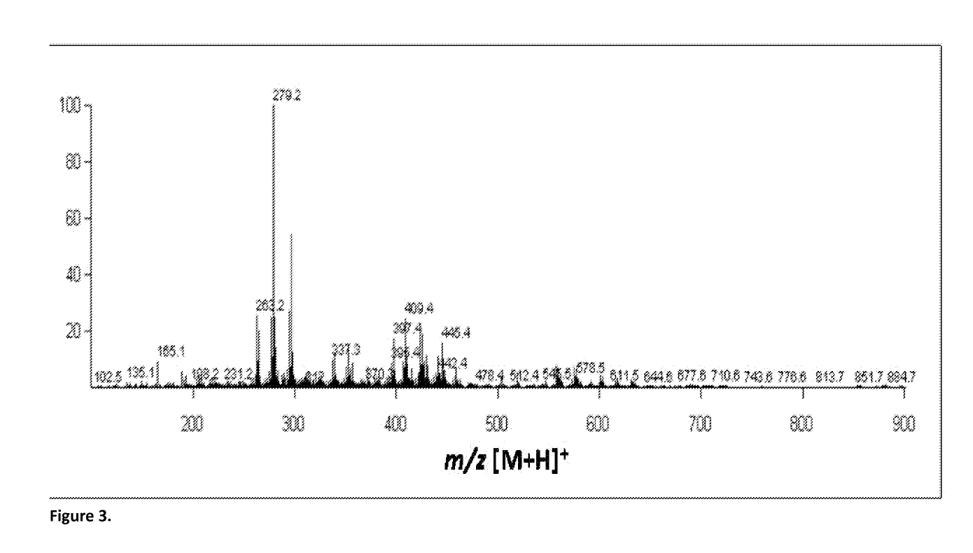

In some embodiments, the SRB extract has a fraction comprising a Direct Analysis in Real Time (DART) mass spectrometry chromatogram of any of FIGS. 2-4.

The aforementioned extracts have certain activity against various therapeutic endpoints, such as COX-1, COX-2 and 5-LOX. In some embodiments, the aforementioned extracts have an IC₅₀ value for COX-1 inhibition of less than 1000 pg/mL. In other embodiments, the IC₅₀ value for COX-1 inhibition is about 1 μg/mL to 500 μg/mL. In other embodiments, the IC₅₀ value for COX-1 inhibition is about 5 μg/mL to 400 μg/mL. In other embodiments, the IC₅₀ value for COX-1 inhibition is about 10 μg/mL to 350 μg/mL. In other embodiments, the IC₅₀ value for COX-1 inhibition is about 10, 20, 30, 40, 50, 60, 70, 80, 90, 100, 150, 200, 250, 300, 310, 320, 330, 340, 350 or 400 μg/mL.

In some embodiments, the SRB extract has an IC₅₀ value for COX-2 inhibition is less than 1000 μg/mL. In some embodiments, the SRB extract has an IC₅₀ value for COX-2 inhibition is about 0.5 μg/mL to 250 μg/mL, 1 μg/mL to 100 μg/mL, or 5 μg/mL to 50 μg/mL. In some embodiments, the IC₅₀ value for COX-2 inhibition is about 5, 10, 15, 20, 25, 30, 35, 40, 45 or 50 μg/mL.

In some embodiments, the SRB extract has an IC₅₀ value for 5-LOX inhibition of less than 1000 μg/mL. In some embodiments, the IC₅₀ value for 5-LOX inhibition is about 1 μg/mL to 500 μg/mL, 10 μg/mL to 500 μg/mL, 25 μg/mL to 400 μg/mL, or 50 μg/mL to 500 μg/mL. In some embodiments, the SRB IC₅₀ value for 5-LOX inhibition is about 50, 60, 70, 80, 90, 100, 125, 150, 175, 200, 225, 250, 275, 300, 325, 350, 374 or 400 μg/mL.

Pharmaceutical Compositions

In some aspects of the invention, pharmaceutical formulations comprising any of the aforementioned and at least one pharmaceutically acceptable carrier are provided.

Compositions of the disclosure comprise extracts of stabilized rice bran in forms such as a paste, powder, oils, liquids, suspensions, solutions, ointments, or other forms, comprising, one or more fractions or sub-fractions to be used as dietary supplements, nutraceuticals, or such other preparations that may be used to prevent or treat various human ailments. The extracts can be processed to produce such consumable items, for example, by mixing them into a food product, in a capsule or tablet, or providing the paste itself for use as a dietary supplement, with sweeteners or flavors added as appropriate. Accordingly, such preparations may include, but are not limited to, rice bran extract preparations for oral delivery in the form of tablets, capsules, lozenges, liquids, emulsions, dry flowable powders and rapid dissolve tablets. Based on the anti-inflammation activities described herein, patients would be expected to benefit from daily dosages in the range of from about 50 mg to about 1000 mg. For example, a capsule comprising about 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, or 250 mg of the extract can be administered once or twice a day to a subject as a prophylactic. Alternatively, in response to severe inflammation, two capsules may be needed every 4 to 6 hours.

In one embodiment, a dry extracted rice bran composition is mixed with a suitable solvent, such as but not limited to water or ethyl alcohol, along with a suitable food-grade material using a high shear mixer and then spray air-dried using conventional techniques to produce a powder having grains of very small rice bran extract particles combined with a food-grade carrier.

In a particular example, rice bran extract composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spray air-drying. Typical amounts are in the range of between about 5 to about 10 liters per kilogram of extracted material. The solution of extract, maltodextrin and ethyl alcohol is spray air-dried to generate a powder with an average particle size comparable to that of the starting carrier material.

In another embodiment, an extract and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extract material is combined with food grade material about one and one-half times by weight of the extract, such as magnesium carbonate having an average particle size of about 20 to 200 micrometers. Inert carriers such as silica having a particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extract is then heated until it flows like a heavy oil. Preferably, it is heated to about 50° C. The heated extract is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extract is introduced into a high shear mixer. The mixture of extract, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired. Alternatively, an extract of Stevia rebaudiana, a very sweet-tasting dietary supplement, can be added instead of, or in conjunction with, a specific sweetening agent (for simplicity, Stevia will be referred to herein as a sweetening agent). After mixing is completed, the mixture is dried using freeze-drying or refractive window drying. The resulting dry flowable powder of extract, magnesium carbonate, silica and optional emulsifying agent and optional sweetener has an average particle size comparable to that of the starting carrier and a predetermined extract.

According to another embodiment, an extract is combined with approximately an equal weight of food-grade carrier such as whey protein, preferably having a particle size of between about 200 to about 1000 micrometers. Inert carriers such as silica having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The extract can be heated until it flows like a heavy oil (preferably heated to about 50° C.). The heated extract is then added incrementally to the whey protein and inert carrier that is being mixed in the food processor-type mixer. The mixing of the extract and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of extract, whey protein, and inert carrier is introduced incrementally into the cold water containing high shear mixer while mixing. Sweetening agents or other taste additives of up to about 5% by weight can be added at this stage if desired. After mixing is completed, the mixture is dried using freeze drying or refractive window drying. The resulting dry flowable powder of extract, whey protein, inert carrier and optional sweetener has a particle size of about 150 to about 700 micrometers and a unique predetermined extract.

In the embodiments where the extract is to be included into an oral fast dissolve tablet as described in U.S. Pat. No. 5,298,261, the unique extract can be used “neat”, that is, without any additional components which are added later in the tablet forming process as described in the patent cited. This method obviates the necessity to take the extract to a dry flowable powder that is then used to make the tablet.

Once a dry extract powder is obtained, such as by the methods discussed herein, it can be distributed for use, e.g., as a dietary supplement or for other uses. In a particular embodiment, the novel extract powder is mixed with other ingredients to form a tableting composition of powder that can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° C. to about 85° C. The dried tablet can then be packaged for distribution Compositions can be in the form of a paste, resin, oil, powder or liquid. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol);preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

Dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

The described extracts may be combined with extracts from other plants such as, but not limited to, varieties of gymnema, turmeric, boswellia, guarana, cherry, lettuce, Echinacea, piper betel leaf, Areca catechu, Muira puama, ginger, willow, suma, kava, horny goat weed, Ginkgo bilboa, maté, garlic, puncture vine, arctic root astragalus, eucommia, gastropodia, and uncaria, or pharmaceutical or nutraceutical agents.

A tableting powder can be formed by adding about 1 to 40% by weight of the powdered extract, with between 30% to about 80% by weight of a dry water-dispersible absorbent such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to about 100 mesh.

Preferably, the tablet exhibits rapid dissolution or disintegration in the oral cavity. The tablet is preferably a homogeneous composition that dissolves or disintegrates rapidly in the oral cavity to release the extract content over a period of about 2 seconds or less than 60 seconds or more, preferably about 3 to about 45 seconds, and most preferably between about 5 to about 15 seconds.

Various rapid-dissolve tablet formulations known in the art can be used. Representative formulations are disclosed, for example, in U.S. Pat. Nos. 5,464,632; 6,106,861; 6,221,392; 5,298,261; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1 and 1.5%. Additional components include maltodextrin (Maltrin, M-500) at between 1 and 5%. These amounts are solubilized in water and used as a starting mixture to which is added the rice bran extraction composition, along with flavors, sweeteners such as Sucralose or Acesulfame K, and emulsifiers such as BeFlora and BeFloraPlus which are extracts of mung bean. A particularly preferred tableting composition or powder contains about 10 to 60% by weight of the extract powder and about 30% to about 60% of a water-soluble diluent.

In a preferred implementation, the tableting powder is made by mixing in a dry powdered form of the various components as described above, e.g., active ingredient (extract), diluent, sweetening additive, flavoring, etc. An overage in the range of about 10% to about 15% of the active extract can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the extract in the form of a dry flowable powder in a single oral dosage is typically in the range of about 40 mg to about 1000 mg. The tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 0.15 inch to about 0.5 inch in diameter and about 0.08 inch to about 0.2 inch in thickness, and has a weight of between about 160 mg to about 1,500 mg. In addition to disk, wafer, or coin shapes, the tablet can be in the form of a cylinder, sphere, cube, or other shapes.

Compositions of unique extract compositions may also comprise extract compositions in an amount between about 10 mg and about 2000 mg per dose.

Methods of Treatment

The aforementioned extracts or pharmaceutical compositions may be administered to a subject in need thereof for treatment or prevention of a variety of disease and conditions. Additionally, the compositions may be administered for the treatment or relief of the symptoms of a variety of conditions. When the symptoms of a disease or condition are treated or prevented, the underlying disease or condition may or may not be treated or prevented, depending on the particular disease or condition.

Accordingly, in some embodiments the present invention provides a method of treating or preventing an inflammatory disorder in a subject comprising administering to a subject in need thereof a therapeutically effective amount of the aforementioned pharmaceutical composition. In some embodiments, the invention provides a method of treating or preventing symptoms of an inflammatory disorder in a subject comprising administering to a subject in need thereof a therapeutically effective amount of aforementioned compositions.

The administration may be oral or topical in some embodiments. For example, the pharmaceutical composition may be formulated as a lotion, cream, ointment, oil, paste or transdermal patch for topical administration. In another embodiment, the composition may be formulated as a functional food, dietary supplement, powder or beverage for administration by ingestion.

The inflammatory disorder may be acute or chronic. In some embodiments, the inflammatory disorder is arthritis, asthma, gout, tendonitis, bursitis, polymyalgia rheumatica or migraine headache. In certain embodiments, the inflammatory disorder is osteoarthritis. In other embodiments, the inflammatory disorder is rheumatoid arthritis.

In some embodiments, the invention provides a method of treating or preventing a neurologic disorder in a subject comprising administering to a subject in need thereof a therapeutically effective amount of any of the aforementioned compositions. In some embodiments, the invention provides a method of treating or preventing symptoms of a neurologic disorder. In some embodiments, the neurologic disorder is selected from the group consisting of Alzheimer's disease, dementia, Parkinson's disease, and migraine headache.

In some embodiments, the invention provides a method of treating or preventing cancer in a subject comprising administering to a subject in need thereof a therapeutically effective amount of any of the aforementioned compositions. In other embodiments, the invention provides a method of treating or preventing symptoms of cancer in a subject. In some embodiments, the cancer is selected from the group consisting of colon cancer, pancreatic cancer, or breast cancer.

### Exemplification

A. Stabilized Rice Bran Feedstocks and Chemicals

Stabilized Rice Bran (SRB) was supplied by Nutracea Inc., USA and stored at room temperature. The SRB was sieved through a 140 mesh screen (100 μm). Liquid CO₂ (purity 99.5%) was supplied by Soxal Co. Ethanol and water (HPLC grade) were purchased from Sigma-Aldrich Co (St. Louis, Mo.).

B. Extraction Procedure

1. Solvent Extraction

A 10 g sample of SRB was extracted in a flask with 150 mL of organic solvents used for plant materials. Solvents of different concentration of ethanol in water like water, 20% (v/v) ethanol, 40% ethanol, 60% ethanol, and 80% ethanol and 100% ethanol were used. The extraction was performed in two, 2-hr stages at temperatures of 20° C. to 60° C. The combined extracts were filtered through Fisher P4 filter paper with a pore size of 4-8 μm, and centrifuged at 2000 rpm for 20 min. The supernatants were collected and evaporated to dryness at 50° C. in a vacuum oven for overnight.

2. Supercritical Carbon Dioxide Extraction

Experiments were performed using an SFT 250 (Supercritical Fluid Technologies, Inc., Newark, Del.) which is designed for pressures and temperatures up to 690 bar and 200° C., respectively. The extraction vessel pressure and temperature are monitored and controlled within to ±3 bar and ±1° C.

A 30 g sample of SRB powder with mesh sizes above 105 μm (measured using a 140 mesh screen) was loaded into a 100-mL extraction vessel. Glass wool was placed at the two ends of the column to avoid any possible carryover of solid material. The oven was preheated to the desired temperature before the packed vessel was loaded. After the vessel was connected into the oven, the extraction system was tested for leakage by pressurizing the system with CO₂ (˜850 psig), and purged. The system was closed and pressurized to the desired extraction pressure using the air-driven liquid pump. The system was then equilibrated for ˜3 min. A sampling vial (40 mL) was weighed and connected to the sampling port. The extraction was started by flowing CO₂ at a rate of ˜10 SLPM (19 g/min), which is controlled by a meter valve. A full factorial extraction design was adopted varying the temperature from 40-80° C. and from 80-500 bar.

C. DART TOF-MS Characterization of Extracts

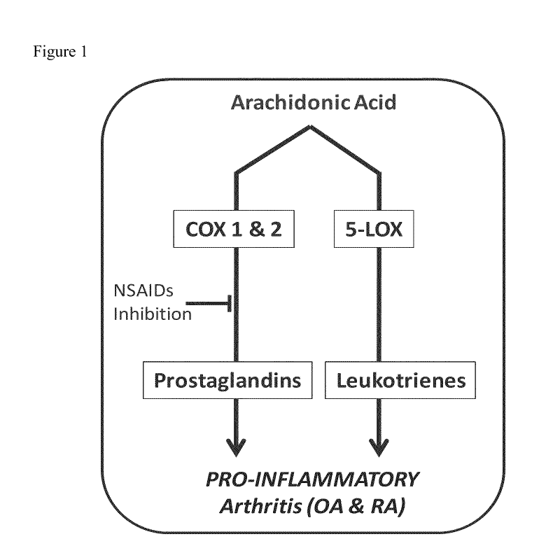

A Jeol DART AccuTOF-MS (Model JMS-T100LC; Jeol USA, Peabody, Mass.) was used for chemical characterization of compounds in the SRB extracts. The DART settings were loaded as follows: DART Needle voltage=3000V; Electrode 1 voltage=150V; Electrode 2 voltage=250 V; Temperature=250° C.; He Flow Rate=2.52 LPM. The following AccuTOF mass spectrometer settings were loaded: Ring Lens voltage=5 V; Orifice 1 voltage=10 V; Orifice 2 voltage=5 V; Peaks voltage=1000 V (for resolution between 100-1000 amu); Orifice 1 temperature was turned off. The samples were introduced by placing the closed end of a borosilicate glass capillary tube into the SRB extracts, and the coated capillary tube was placed into the DipIT™ sample holder providing a uniform and constant surface exposure for ionization in the He plasma. The SRB extract was allowed to remain in the He plasma stream until signal was observed in the total-ion-chromatogram (TIC).The sample was removed and the TIC was brought down to baseline levels before the next sample was introduced. A polyethylene glycol 600 (Ultra Chemicals, Kingston R.I.) was used as an internal calibration standard giving mass peaks throughout the desired range of 100-1000 amu. The DART mass spectrum of each SRB extract was searched against a proprietary chemical database and used to identify many of the compounds present in the extracts. Search criteria were held to the [M+H]⁺ ions to within 10 mmu of the calculated masses. DART mas spectrum of SRB Extract 1, SRB Extract 2, and SRB Extract 3 are shown in FIGS. 1, 2, and 3, respectively, with the X-axis showing the mass distribution (100-1000 amu) and the Y-axis showing the relative abundances of each chemical species detected. The DART TOF-MS of SRB Extract 1 an extract enriched in COX-1 and COX-2 inhibitory activity, but absent 5-LOX inhibition activity, is shown in FIG. 1. Table 1 lists the compounds identified in the SRB Extract 1.

TABLE 1
Summary of the compounds identified in SRB Extract 1 by DART TOF-MS.

| | Measured | Calculated | Difference | Relative |
|---|---|---|---|---|
| Compund Name | Mass | Mass | (amu) | Abundance (%) |
| aminobutyric acid | 104.0709 | 104.0711 | −0.0002 | 31.3429 |
| 2-ethlpyrazine | 109.0763 | 109.0765 | −0.0002 | 5.7722 |
| norcamphor/heptadienal | 111.0892 | 111.081 | 0.0082 | 7.4721 |
| histamine | 112.0867 | 112.0874 | −0.0008 | 20.0377 |
| proline | 116.0706 | 116.0711 | −0.0005 | 31.0365 |
| levulinic acid | 117.0525 | 117.0551 | −0.0026 | 0.4597 |
| valine | 118.0872 | 118.0868 | 0.0004 | 18.6825 |
| L-threonine | 120.0676 | 120.066 | 0.0016 | 3.1124 |
| conyrin | 122.0835 | 122.0931 | −0.0096 | 2.1683 |
| 2-ethyl-3-methylpyrazine | 123.0909 | 123.0922 | −0.0013 | 45.2772 |
| pyrogallol/phlorglucinol | 127.0416 | 127.0395 | 0.0021 | 3.9529 |
| leucine | 132.1019 | 132.1024 | −0.0005 | 14.7282 |
| ocimene/camphene/adamantane | 137.1076 | 137.1078 | −0.0003 | 39.123 |
| histidinol | 142.101 | 142.098 | 0.003 | 14.4119 |
| octalactone | 143.1021 | 143.1072 | −0.0051 | 5.0493 |
| 3-hydroxy-2,3 dihydromaltol | 145.0504 | 145.0501 | 0.0002 | 13.1694 |
| lysine | 147.0939 | 147.0922 | 0.0016 | 2.4956 |
| 4-hydroxyisoleucine | 148.0963 | 148.0973 | −0.0011 | 11.177 |
| cuminaldehyde | 149.1022 | 149.0966 | 0.0056 | 10.6597 |
| carvacrol/thymol/cymenol | 151.1223 | 151.1235 | −0.0012 | 24.6854 |
| cineole/borneol/pulegol | 155.1365 | 155.1436 | −0.0071 | 16.0232 |
| arecoline/hydroxytropinone | 156.108 | 156.1024 | 0.0056 | 19.6283 |
| nonalactone | 157.1311 | 157.1228 | 0.0083 | 0.9857 |
| betonicine/acetyl valine | 160.1007 | 160.0973 | 0.0034 | 19.8363 |
| tryptamine | 161.1074 | 161.1078 | −0.0004 | 6.4302 |
| carnitine, L- | 162.109 | 162.113 | −0.004 | 10.3164 |
| acetylthiocholine | 163.103 | 163.1031 | −0.0002 | 16.2176 |
| N-phenylmorpholine | 164.1058 | 164.1075 | −0.0017 | 13.1874 |
| jasmone | 165.1347 | 165.1279 | 0.0068 | 13.8752 |
| hordenine | 166.1155 | 166.1232 | −0.0077 | 26.1225 |
| L-methylhistidine | 170.1019 | 170.0929 | 0.0089 | 14.6776 |
| nonandedioic acid anhydride | 171.1097 | 171.1021 | 0.0076 | 5.7877 |
| n-acetyl-DL-leucine | 174.1202 | 174.113 | 0.0072 | 14.8065 |
| arginine | 175.1264 | 175.1195 | 0.0068 | 3.5287 |
| 4-dimethylaminocinnamaldehyde | 176.1137 | 176.1075 | 0.0062 | 9.0433 |
| 2-pentanone, 4-methyl-4-phen | 177.1221 | 177.1279 | −0.0058 | 8.6586 |
| salsolinol | 180.1065 | 180.1024 | 0.0041 | 35.3133 |
| 2(4H)-benzofuranone, 5,6,7,7 | 181.115 | 181.1228 | −0.0078 | 27.7225 |
| 3-methyl-2-butenoic acid, 2- | 185.1168 | 185.1177 | −0.0009 | 8.0583 |
| tetrahydrofurylmethyl ester | | | | |
| DL-eleagnin | 187.1202 | 187.1235 | −0.0033 | 6.1334 |
| Epiloliolide | 197.1281 | 197.1182 | 0.0099 | 22.2178 |
| 1,4-cineole | 203.1377 | 203.1436 | −0.0059 | 4.2732 |
| isopilocarpine/philocarpine | 209.1385 | 209.129 | 0.0095 | 18.7459 |
| (S)-(+)-carvone acetate | 211.1429 | 211.1334 | 0.0094 | 18.9403 |
| 2-nitrocyclopentanemethanol | 216.1371 | 216.1388 | −0.0018 | 16.7 |
| proposed compound 3/2-(3-hyd | 219.1319 | 219.1385 | −0.0066 | 11.3562 |
| costunolide | 233.1452 | 233.1541 | −0.0089 | 12.3094 |
| retene | 235.1472 | 235.1487 | −0.0015 | 9.2535 |
| cyclooctyl propylphosphonofl | 237.1465 | 237.1419 | 0.0046 | 12.7133 |
| huperazine A | 243.1508 | 243.1497 | 0.001 | 5.8779 |
| panaxynol | 245.1844 | 245.1905 | −0.0061 | 6.2005 |
| parthenolide | 249.1525 | 249.149 | 0.0035 | 12.9955 |
| palmitic acid | 257.249 | 257.248 | 0.001 | 4.2469 |
| panaxydol | 261.1781 | 261.1854 | −0.0073 | 10.3779 |
| 9,12,15-octadecatrien-1-ol | 265.2513 | 265.2531 | −0.0019 | 37.917 |
| 17-estradiol | 273.1927 | 273.1854 | 0.0073 | 6.2143 |
| octadecatrienoic acid | 279.2321 | 279.2324 | −0.0003 | 100 |
| octadecadienoic acid | 281.2471 | 281.248 | −0.001 | 78.0647 |
| octadecenoic acid | 283.2634 | 283.2637 | −0.0003 | 38.0737 |
| tropicamide | 285.167 | 285.1603 | 0.0066 | 2.6228 |
| androstenedione | 287.1971 | 287.2011 | −0.004 | 5.9017 |
| 7-shogaol | 291.189 | 291.196 | −0.007 | 9.2696 |
| nordihydrocapsaicin | 294.2125 | 294.2069 | 0.0055 | 7.5274 |
| cryptotanshinone | 299.1677 | 299.1647 | 0.003 | 1.8493 |
| lauric acid, 2-butoxyethyl ester | 301.2759 | 301.2742 | 0.0017 | 14.5412 |
| 10-paradol | 307.2184 | 307.2273 | −0.0089 | 4.9474 |
| dihydrocapsaicin | 308.2261 | 308.2225 | 0.0036 | 7.406 |
| octadecenoic acid ethyl ester | 311.2932 | 311.295 | −0.0018 | 8.4539 |
| progesterone | 315.2323 | 315.2324 | −0.0001 | 4.6396 |
| cafestol | 317.2059 | 317.2116 | −0.0057 | 4.6189 |
| galanolactone/aframodial | 319.2242 | 319.2273 | −0.0032 | 7.4937 |
| homocapsaicin | 320.2168 | 320.2226 | −0.0058 | 5.8132 |
| 8-gingerdione | 321.2089 | 321.2066 | 0.0023 | 3.5195 |
| homodihydrocapsaicin | 322.2406 | 322.2382 | 0.0024 | 6.2174 |
| 8-gingerol/rapanone | 323.222 | 323.2222 | −0.0002 | 3.2185 |
| crocetin/geranoxy methoxycoumarin | 329.1738 | 329.1753 | −0.0015 | 1.0941 |
| 14-deoxy-11,12- | 334.2219 | 334.2144 | 0.0075 | 4.3115 |
| didehydroandrgrapholide | | | | |
| deoxy-andrographolide | 335.2312 | 335.2222 | 0.009 | 5.4907 |
| pregnanetriol | 337.2749 | 337.2742 | 0.0007 | 13.9251 |
| magnoflorine | 343.1836 | 343.1783 | 0.0053 | 1.046 |
| 12-shogaol | 361.2806 | 361.2743 | 0.0063 | 5.4468 |
| cinobufotalin | 363.2741 | 363.2688 | 0.0053 | 3.031 |
| lithocholic acid | 377.2955 | 377.3055 | −0.01 | 4.5103 |
| pentacosanoic acid | 383.3795 | 383.3889 | −0.0094 | 12.7942 |
| octyl phthalate | 391.2941 | 391.2848 | 0.0093 | 20.8872 |
| fucosterol/sitosterone/spinasterol | 413.384 | 413.3783 | 0.0057 | 5.2398 |
| calcitriol/sarsapogenin | 417.3273 | 417.3368 | −0.0096 | 5.7665 |
| lanosterol/amyrin/lupeol | 427.3881 | 427.394 | −0.0059 | 9.8247 |
| cholesteryl acetate | 429.374 | 429.3732 | 0.0008 | 14.7349 |
| cerevisterol | 431.3503 | 431.3525 | −0.0022 | 5.3071 |
| methoxycerevisterol | 445.3712 | 445.3682 | 0.0031 | 17.3784 |
| celastrol | 451.2929 | 451.2848 | 0.008 | 0.9092 |
| ursolic/oleanolic/boswellic acids | 457.3731 | 457.3682 | 0.005 | 5.4556 |
| jujubogenin/bacoside A | 473.3586 | 473.3631 | −0.0044 | 2.1729 |
| cholesteryl benzoate | 491.3937 | 491.3889 | 0.0047 | 3.5586 |
| gymnestrogenin/gymnemagenin | 507.3755 | 507.3686 | 0.0069 | 2.1238 |

The DART TOF-MS fingerprint of SRB Extract 2, an extract possessing 5-LOX inhibitory activity as well as activity against both the COX-1 and COX-2 enzymes is shown in FIG. 2. Table 2 lists the chemicals identified in SRB extract 2 by DART TOF-MS.

TABLE 2
Summary of the compounds identified in SRB Extract 2 by DART
TOF-MS.

| | Measured | Calculated | Difference | Relative |
|---|---|---|---|---|
| Compound Name | Mass | Mass | (amu) | Abundance (%) |
| valeric/methylbutyric acid | 103.0696 | 103.0759 | −0.0063 | 0.1226 |
| ethylbenzene | 107.0801 | 107.0861 | −0.006 | 0.4146 |
| 2-acetylpyrrole | 110.0583 | 110.0606 | −0.0023 | 0.0206 |
| povidone | 112.0762 | 112.0762 | 0 | 0.0437 |
| hexanoic acid/butyl acetate | 117.084 | 117.0915 | −0.0075 | 0.6863 |
| pseudocumene | 121.0987 | 121.1017 | −0.003 | 0.2749 |
| 2,6-dimethylanilene/conyrin | 122.1004 | 122.0969 | 0.0035 | 0.1068 |
| 2-acetylpyrazine | 123.0582 | 123.0558 | 0.0024 | 0.3772 |
| 6-methyl-5-hepten-2-one | 127.1101 | 127.1123 | −0.0022 | 0.241 |
| ornithine | 133.0986 | 133.0977 | 0.0009 | 0.4844 |
| p-cymene | 135.1161 | 135.1174 | −0.0013 | 1.3302 |
| diethylpyrazine | 137.1138 | 137.1078 | 0.0059 | 0.5157 |
| histidinol | 143.1056 | 143.1072 | −0.0016 | 0.9858 |
| lysine | 147.1162 | 147.1133 | 0.0029 | 0.2901 |
| nornicotine | 149.1092 | 149.1078 | 0.0014 | 1.8341 |
| 1-methyl-3-phenylpropylamine | 150.1316 | 150.1282 | 0.0033 | 0.4354 |
| 2-butyl-3-methylpyrazine | 151.1214 | 151.1235 | −0.0021 | 0.7076 |
| norpseudophedrine | 152.1143 | 152.1075 | 0.0068 | 0.3935 |
| adonitol/arabitol | 153.0755 | 153.0763 | −0.0008 | 0.7009 |
| pseudopelletierine | 154.1248 | 154.1232 | 0.0016 | 0.6389 |
| methyl-2-octynoate | 155.1066 | 155.1072 | −0.0006 | 1.341 |
| 2,6-tropanediol | 158.123 | 158.1181 | 0.0049 | 0.3425 |
| tryptamine | 161.1339 | 161.1416 | −0.0077 | 0.3462 |
| DL-anabasine | 163.1274 | 163.1235 | 0.0039 | 1.053 |
| jasmone | 165.1328 | 165.1279 | 0.0049 | 9.0795 |
| 2,4-hexadienoic acid isobutylamide | 168.1295 | 168.1388 | −0.0093 | 0.4195 |
| lupinine | 170.1489 | 170.1545 | −0.0056 | 0.1365 |
| (+)-1S,2S—N-methylpseudoephe | 180.1363 | 180.1388 | −0.0026 | 0.3658 |
| Acetyllaburnine | 184.1362 | 184.1338 | 0.0024 | 0.3901 |
| pinonic acid | 185.1248 | 185.1177 | 0.0071 | 0.2592 |
| Nonanedioic acid diamide | 187.1449 | 187.1447 | 0.0002 | 0.3116 |
| damascone | 193.1623 | 193.1592 | 0.003 | 3.7552 |
| dehydrocurcumene | 201.1645 | 201.1643 | 0.0002 | 0.4781 |
| curcumene | 203.1819 | 203.18 | 0.0018 | 2.9609 |
| zingiberene/(Z,E)-a-farnesene | 205.1925 | 205.1956 | −0.0031 | 3.7418 |
| valeric acid phenylethyleste | 207.1431 | 207.1385 | 0.0046 | 2.8396 |
| carvylacetate | 209.1577 | 209.1541 | 0.0036 | 3.9073 |
| isobornyl propionate | 211.1705 | 211.1698 | 0.0006 | 1.6825 |
| benzene, 1-(3-cyclopentylpro | 217.1873 | 217.1956 | −0.0083 | 2.6196 |
| caryophellene oxide | 221.1859 | 221.1905 | −0.0046 | 2.6485 |
| 2,2,6-trimethyl-1-(3-methylb | 223.1657 | 223.1698 | −0.0041 | 1.6978 |
| vellerdiol | 237.1935 | 237.1854 | 0.0081 | 2.0808 |
| Z,Z-7,11-hexadecadien-1-ol | 239.239 | 239.2375 | 0.0014 | 1.2259 |
| heptadecane | 241.2964 | 241.2895 | 0.0069 | 0.0548 |
| matrine | 249.1896 | 249.1967 | −0.0072 | 1.8841 |
| Farnesatrienetriol | 255.2011 | 255.196 | 0.0051 | 0.8327 |
| Farnesylacetone | 263.2357 | 263.2375 | −0.0018 | 25.2437 |
| octadecatrienol | 265.2543 | 265.2531 | 0.0012 | 19.8687 |
| hydroxypalmitic acid | 273.2361 | 273.2429 | −0.0068 | 3.3965 |
| octadecatrienoic acid | 279.2334 | 279.2324 | 0.001 | 100 |
| stearolic acid | 281.2484 | 281.248 | 0.0004 | 14.219 |
| oleic acid | 283.2643 | 283.2637 | 0.0005 | 5.6104 |
| hydroxyoctadecatrienoic acid | 295.2319 | 295.2273 | 0.0046 | 27.1143 |
| hydroxyoctadecenoic acid | 299.2655 | 299.2586 | 0.0069 | 4.4521 |
| abietic acid | 303.2296 | 303.2324 | −0.0028 | 2.3247 |
| arachidonic acid | 305.2401 | 305.248 | −0.0079 | 2.4402 |
| epoxyhydroxyoctadecanoic acid | 313.2697 | 313.2742 | −0.0046 | 4.5871 |
| 3′,4′,7-trimethoxyflavone | 315.1181 | 315.1232 | −0.0051 | 0.0109 |
| allopregnendione | 317.2427 | 317.248 | −0.0053 | 2.2374 |
| 2-chloroethyl palmitate | 319.2388 | 319.2404 | −0.0016 | 2.4681 |
| incensole oxide | 323.2672 | 323.2586 | 0.0085 | 1.9118 |
| ajmaline | 327.2065 | 327.2072 | −0.0007 | 0.9321 |
| hydroxyprogesterone/DHEA acetate | 331.2288 | 331.2273 | 0.0015 | 0.7304 |
| 17a-hydroxypregnenolone | 335.2565 | 335.2586 | −0.0021 | 2.7721 |
| pregnanetriol | 337.2776 | 337.2742 | 0.0034 | 9.9278 |
| urushiol I | 349.3112 | 349.3106 | 0.0006 | 3.3578 |
| 10-gingerdiol | 353.275 | 353.2692 | 0.0058 | 13.5583 |
| chlorogenic acid/scopolin | 355.1067 | 355.1029 | 0.0037 | 0.006 |
| sweroside | 359.1384 | 359.1342 | 0.0042 | 0.0035 |
| 6-methyl-16-dehydropregnenol | 371.2684 | 371.2586 | 0.0098 | 7.2033 |
| cholestenone/cholecalciferol | 385.3525 | 385.347 | 0.0055 | 1.2334 |
| brassicasterol/ergostadienol | 399.3656 | 399.3627 | 0.0029 | 2.6433 |
| solanine D | 400.3654 | 400.3579 | 0.0074 | 1.2241 |
| delta-tocopherol | 403.349 | 403.3576 | −0.0086 | 1.8963 |
| mogroside backbone - 4H2O | 405.3475 | 405.3522 | −0.0046 | 2.7262 |
| squalene | 411.3967 | 411.3991 | −0.0024 | 8.0438 |
| fucosterol/sitosterone/spinasterol | 413.3805 | 413.3783 | 0.0021 | 3.3178 |
| mogroside backbone - 3H2O | 423.3721 | 423.3627 | 0.0094 | 19.0339 |
| amyrenone/lupenone | 425.3765 | 425.3783 | −0.0018 | 18.6519 |
| lanosterol/cycloartenol | 427.3861 | 427.394 | −0.0079 | 9.0533 |
| cholesteryl acetate | 429.3724 | 429.3732 | −0.0008 | 11.2942 |
| vitamin E | 431.3794 | 431.3889 | −0.0095 | 3.1676 |
| mogroside backbone - 2H2O | 441.3756 | 441.3733 | 0.0023 | 10.6668 |
| uvaol/erythrodiol/betulin | 443.3862 | 443.3889 | −0.0027 | 4.8768 |
| methoxycerevisterol | 445.368 | 445.3682 | −0.0002 | 15.6748 |
| vitamin K1(phytonadione) | 451.3577 | 451.3576 | 0 | 1.4888 |
| ursonic acid/dehydroboswellic acid | 455.3529 | 455.3525 | 0.0004 | 2.6857 |
| ursolic/oleanolic/boswellic acids | 457.3708 | 457.3682 | 0.0026 | 3.6601 |
| soyasapogenol B | 458.371 | 458.376 | −0.005 | 1.4913 |
| ganoderic acid D/M | 469.3276 | 469.3318 | −0.0042 | 0.2365 |
| keto boswellic acid | 471.3564 | 471.3474 | 0.009 | 1.4833 |
| jujubogenin/bacoside A | 473.3564 | 473.3631 | −0.0067 | 1.6613 |
| soyasapogenol A | 474.3746 | 474.3709 | 0.0037 | 0.743 |
| Gymnemasaponin II - 2 Glc | 475.3796 | 475.3787 | 0.0008 | 1.2492 |
| panaxatriol/protopanaxatriol | 477.3944 | 477.3944 | 0 | 0.9131 |
| keto boswellic acid | 487.3788 | 487.3787 | 0 | 0.5714 |
| adhyperforin | 551.4087 | 551.41 | −0.0014 | 0.0742 |
| cafestol palmitate | 555.4493 | 555.4413 | 0.008 | 1.2488 |

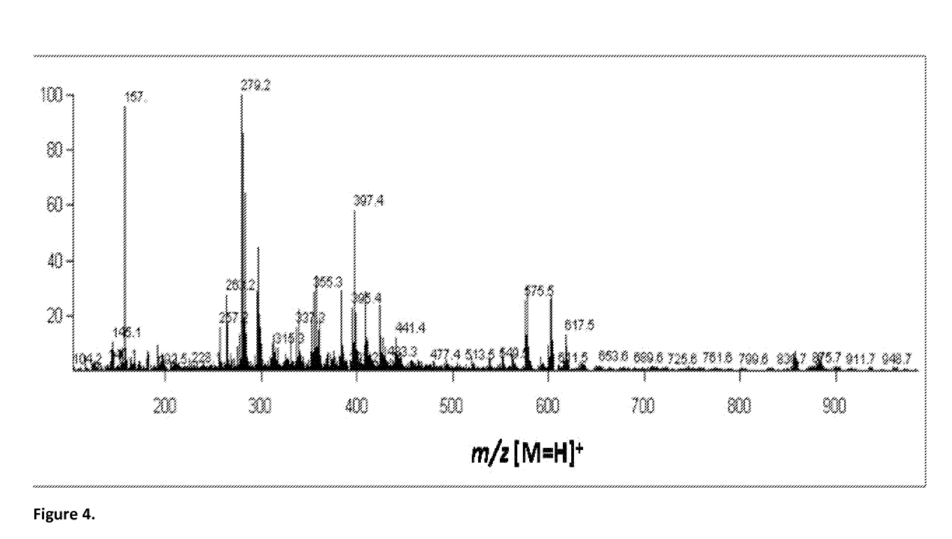

The DART TOF-MS fingerprint of SRB Extract 3, an extract which represents a blend of SRB Extract 1 and SRB Extract 2 in a ratio of 1 part SRB extract 1 to 7 parts SRB Extract 2 (wt/wt) is shown in FIG. 4. This extract blend combines the greatest biological activities of SRB Extract 1 and SRB Extract 2 and is enriched in COX-1, COX-2, and 5-LOX inhibitory activities. Table 3 lists the chemicals identified in SRB Extract 3 by DART TOF-MS.

TABLE 3
Summary of the chemicals identified in SRB Extract 3 by DART TOF-MS.

| | Measured | Calculated | Difference | Relative |
|---|---|---|---|---|
| Compound Name | Mass | Mass | (amu) | Abundance (%) |
| aminobutyric acid | 104.0739 | 104.0711 | 0.0028 | 2.4605 |
| 2-ethylpyrazine | 109.0765 | 109.0765 | 0.0000 | 0.4969 |
| norchamphor/heptadienal | 110.0728 | 110.0736 | −0.0009 | 0.4518 |
| povidone | 112.0861 | 112.0762 | 0.0099 | 1.1385 |
| proline | 116.0725 | 116.0711 | 0.0014 | 5.2037 |
| levulinic acid | 117.0555 | 117.0551 | 0.0004 | 0.6368 |
| Betaine | 118.0772 | 118.0868 | −0.0096 | 0.3564 |
| L-threonine | 120.0645 | 120.0660 | −0.0015 | 0.6051 |
| 2-Phenylethanol | 123.0871 | 123.0810 | 0.0061 | 1.7635 |
| niacin | 124.0417 | 124.0398 | 0.0019 | 2.5261 |
| 6-methyl-5-hepten-2-one | 127.0421 | 127.0395 | 0.0026 | 3.2367 |
| Baikiain | 128.0752 | 128.0711 | 0.0041 | 1.0077 |
| azulene | 129.0675 | 129.0704 | −0.0029 | 0.6407 |
| leucine | 132.1024 | 132.1024 | 0.0000 | 2.3971 |
| Arabinan | 133.0565 | 133.0501 | 0.0064 | 0.757 |
| ocimene/camphene/adamantane | 137.0993 | 137.0926 | 0.0068 | 1.4181 |
| Methyl ester Baikiain | 142.0951 | 142.0868 | 0.0083 | 0.6839 |
| histidinol | 143.1069 | 143.1072 | −0.0003 | 2.8629 |
| 1,4-Dihydroxy-2-cyclopentene-1- | 144.0634 | 144.0660 | −0.0026 | 4.0356 |
| carboxamide | | | | |
| 1-methyl-5-Fluoro-2,4(1H,3H)- | 145.0513 | 145.0413 | 0.0100 | 10.2898 |
| pyrimidinedione | | | | |
| lysine | 147.0611 | 147.0657 | −0.0046 | 0.4211 |
| albizzhn | 148.0820 | 148.0722 | 0.0098 | 0.777 |
| O-Carbamoylserine | 149.0640 | 149.0562 | 0.0078 | 1.5101 |
| Hydrazide | 152.0860 | 152.0824 | 0.0036 | 0.9671 |
| N-Acetylhistamine | 154.1003 | 154.0980 | 0.0023 | 2.2859 |
| 5-Hydroxy-3-isopropyl-2- | 155.1074 | 155.1072 | 0.0002 | 7.9215 |
| cyclohexen-1-one | | | | |
| arecoline/hydroxytropinone | 156.1069 | 156.1024 | 0.0045 | 2.294 |
| Zymonic acid | 159.0323 | 159.0293 | 0.0030 | 8.102 |
| betonicine | 160.1067 | 160.0973 | 0.0094 | 3.4168 |
| tryptamine | 161.0422 | 161.0450 | −0.0028 | 0.3883 |
| L-2-aminoadipic acid | 162.0845 | 162.0766 | 0.0079 | 1.1537 |
| glyogen | 163.0657 | 163.0606 | 0.0051 | 3.4991 |
| 3-Phenyloxiranecarboxylic acid | 164.0775 | 164.0711 | 0.0064 | 4.8881 |
| 4-(Ethylamino)benzoic acid | 166.0941 | 166.0868 | 0.0073 | 2.493 |
| 1,2-Diethoxybenzene | 167.1084 | 167.1072 | 0.0012 | 2.5424 |
| Nonanedioic acid anhydride | 171.0976 | 171.1021 | −0.0045 | 1.8147 |
| citrulline | 176.1090 | 176.1035 | 0.0055 | 0.4806 |
| 6-Amino-4,5-dihydroxy-3- | 177.0961 | 177.0875 | 0.0086 | 1.4041 |
| piperidinecarboxylic acid | | | | |
| 2-Amino-2,3-dideoxy-ribo-hexose | 178.0998 | 178.1079 | −0.0081 | 0.9747 |
| Me glycoside | | | | |
| 1-Amino-1-deoxyfructose | 180.0918 | 180.0872 | 0.0047 | 4.0148 |
| 2-Amino-2-deoxymannitol | 182.1032 | 182.1028 | 0.0003 | 3.1854 |
| Barnol | 183.1086 | 183.1021 | 0.0065 | 5.7675 |
| Barbital | 185.1000 | 185.0926 | 0.0074 | 1.8721 |
| N-Ethylbenzenesulfonamide | 186.0640 | 186.0588 | 0.0051 | 0.2169 |
| Epilupinine | 186.1551 | 186.1494 | 0.0057 | 0.408 |
| 5-Fluoro-2,4(1H,3H)- | 187.0822 | 187.0883 | −0.0061 | 0.8562 |
| pyrimidinedione | | | | |
| Nonanedioic acid diamide | 188.0971 | 188.0923 | 0.0048 | 1.9201 |
| 2-Deoxy-erythro-pentose Me | 189.1139 | 189.1127 | 0.0012 | 0.9383 |
| glycoside, 3,4-O-isopropylidene | | | | |
| castanospermine | 190.1014 | 190.1079 | −0.0065 | 0.6899 |
| damascone | 193.1592 | 193.1592 | 0.0000 | 4.8934 |
| 2-Methylpropyl 4-aminobenzoate | 194.1146 | 194.1181 | −0.0034 | 2.0927 |
| 2,3,4-trimethyl-arabinitol | 195.1328 | 195.1232 | 0.0096 | 3.1128 |
| Epiloliolide | 197.1267 | 197.1177 | 0.0089 | 5.7445 |
| 2-Amino-1-(3,4- | 198.1198 | 198.1130 | 0.0068 | 3.6462 |
| dimethoxyphenyl)ethanol | | | | |
| 1-Phenoxy-2-phenylethane | 199.1141 | 199.1123 | 0.0018 | 3.5459 |
| 2-(2,4-hexadiynylidene)-1,6- | 201.0972 | 201.0915 | 0.0057 | 0.6527 |
| dioxaspiro[4.4]non-3-ene | | | | |
| Zanthonitrile | 202.1328 | 202.1232 | 0.0097 | 0.5658 |
| 3-Amino-2,3,6-trideoxy-arabino- | 204.1238 | 204.1236 | 0.0002 | 0.2034 |
| hexose 4-Me, N-Ac | | | | |
| 2-Amino-3a,5,6,6a-tetrahydro-4- | 205.0902 | 205.0824 | 0.0077 | 3.1563 |
| (hydroxymethyl)-4H- | | | | |
| cyclopentoxazole-4,5,6-triol | | | | |
| 2-Amino-2,3-dideoxy-ribo-hexose | 206.1066 | 206.1028 | 0.0038 | 1.3217 |
| N-Ac | | | | |
| Cytisine N-oxide | 207.1211 | 207.1133 | 0.0078 | 1.4139 |
| 2-Amino-2-deoxygalactose, 3,4-Di- | 208.1142 | 208.1185 | −0.0042 | 1.6768 |
| Me | | | | |
| carvylacetate | 209.1518 | 209.1541 | −0.0023 | 3.5783 |
| 9,10-Epoxytetrahydroedulan | 211.1625 | 211.1698 | −0.0073 | 5.5854 |
| N1,N3-Di-Methyl Barbital | 213.1265 | 213.1239 | 0.0026 | 2.6769 |
| 4,6-Tetradecadiene-8,10,12-triyn-1- | 215.1139 | 215.1072 | 0.0068 | 1.4883 |
| ol; 4,5-Epoxy-6-tetradecene-8,10,12- | | | | |
| triyn-1-ol | | | | |
| Nonanedioic acid dimethyl ester | 217.1448 | 217.1440 | 0.0008 | 1.0033 |
| 3-Amino-2,3,6-trideoxy-arabino- | 218.1378 | 218.1392 | −0.0014 | 0.7832 |
| hexose Me glycoside, 4-Me, N-Ac | | | | |
| abrine | 219.1216 | 219.1133 | 0.0083 | 0.859 |
| vitamin B5 | 220.1220 | 220.1185 | 0.0036 | 1.536 |
| 9-acetylphenanthrene | 221.1051 | 221.0966 | 0.0085 | 0.8129 |
| 2-Acetamido-2-deoxyglucose | 222.1077 | 222.0977 | 0.0100 | 0.7515 |
| 3-Methylbutyl 4-methoxybenzoate | 223.1424 | 223.1334 | 0.0091 | 1.3976 |
| Epiguaymasol | 225.1559 | 225.1490 | 0.0068 | 4.3033 |
| Macromerine | 226.1489 | 226.1443 | 0.0046 | 0.9275 |
| Arthropsatriol B | 227.1375 | 227.1283 | 0.0092 | 2.5268 |
| 2,5-Epidioxy-2-hydroxy-5-isopropyl- | 229.1370 | 229.1440 | −0.0070 | 2.2033 |
| 3-nonen-8-one | | | | |
| Melatonin | 233.1294 | 233.1290 | 0.0004 | 2.8342 |
| Erythrinarbine | 234.1227 | 234.1130 | 0.0097 | 0.8227 |
| 2,6-Diamino-2,6-dideoxyidose Me | 235.1206 | 235.1294 | −0.0087 | 1.0487 |
| glycoside, 2N-Ac | | | | |
| 6-Deoxy-5-C-methyl-lyxo-hexose 4- | 236.1231 | 236.1134 | 0.0097 | 0.8639 |
| Me, 3-carbamoyl | | | | |
| Fructose, 9CI, 8CI Butyl glycoside | 237.1244 | 237.1338 | −0.0094 | 1.6476 |
| 11-hexadecyn-1-ol | 239.2390 | 239.2375 | 0.0015 | 3.7761 |
| Ophidine | 241.1380 | 241.1300 | 0.0080 | 1.6622 |
| Bauhinol C | 243.1398 | 243.1385 | 0.0013 | 1.3249 |
| 3-Amino-2,3,6-trideoxy-arabino- | 246.1344 | 246.1341 | 0.0003 | 1.7972 |
| hexose Me glycoside, N,O-di-Ac | | | | |
| 2,6-Dideoxy-3-C-methyl-arabino- | 247.1202 | 247.1181 | 0.0020 | 1.6925 |
| hexose 1,4-Di-Ac | | | | |
| 4-Epiphyllanthine | 248.1249 | 248.1286 | −0.0037 | 1.2317 |
| 1,2,3,10,11,11a-Hexahydro-2- | 249.1319 | 249.1239 | 0.0080 | 1.7636 |
| hydroxy-11-methoxy-5H- | | | | |
| pyrrolo[2,1-c][1,4]benzodiazepin-5- | | | | |
| one | | | | |
| 2-Acetamido-2-deoxyglucose 3,4-Di- | 250.1388 | 250.1290 | 0.0097 | 0.9558 |
| Me | | | | |
| 4-Epilegionamic acid | 251.1315 | 251.1243 | 0.0072 | 2.0557 |
| 2-Phenylethyl 3-phenyl-2-propenoate | 253.1285 | 253.1228 | 0.0056 | 1.0753 |
| 2-Amino-2-deoxyglucose 2,3- | 254.1260 | 254.1240 | 0.0020 | 0.8236 |
| Dihydroxypropyl glycoside | | | | |
| Farnesatrienetriol | 255.2230 | 255.2297 | −0.0067 | 5.0757 |
| palmitic acid | 257.2490 | 257.2480 | 0.0010 | 15.6197 |
| 14(5→6)-Abeo-1,5,9- | 259.1387 | 259.1334 | 0.0053 | 2.6241 |
| furanoeremophilatriene-9,14-diol. | | | | |
| 14-Hydroxydemethylcacalohastine | | | | |
| 1-Amino-1-deoxyfructose 2,3:4,5- | 260.1422 | 260.1498 | −0.0076 | 0.9962 |
| Di-O-isopropylidene | | | | |
| 13A-Hydroxy, 5,6-didehydro | 261.1686 | 261.1603 | 0.0083 | 2.8799 |
| Multiflorine | | | | |
| Acrifoline | 262.1838 | 262.1807 | 0.0031 | 1.7447 |
| Farnesylacetone | 263.2367 | 263.2375 | −0.0007 | 27.1309 |
| Octadecatrienol | 265.2531 | 265.2531 | 0.0000 | 16.6139 |
| 5-Hydroxytryptamine benzyl ether | 267.1560 | 267.1497 | 0.0063 | 1.9456 |
| 2,6-Diamino-2,6-dideoxyglucose | 269.1552 | 269.1501 | 0.0050 | 3.546 |
| 1,6,11-Farnesatriene-3,5,8,10-tetrol | 271.1998 | 271.1909 | 0.0089 | 3.9726 |
| Nematophin | 273.1622 | 273.1603 | 0.0020 | 1.4074 |
| (−)-Normaritidine | 274.1497 | 274.1443 | 0.0054 | 0.7898 |
| Epilobscurinol | 276.1906 | 276.1963 | −0.0057 | 2.2137 |
| 12-Phenyldodecanoic acid | 277.2167 | 277.2167 | 0.0000 | 12.8687 |
| Lycopodium Base V | 278.2181 | 278.2120 | 0.0061 | 4.1435 |
| octadecatrienoic acid | 279.2321 | 279.2324 | −0.0002 | 100 |
| 9,12-octadecadienoic acid | 281.2473 | 281.2480 | −0.0007 | 86.0096 |
| Epilachnene | 282.2532 | 282.2433 | 0.0099 | 19.2178 |
| Oxacyclononadecan-2-one | 283.2628 | 283.2637 | −0.0009 | 64.5671 |
| ethylpalmitate | 285.2761 | 285.2793 | −0.0032 | 4.661 |
| 17-Hydroxyandrosta-1,4-dien-3-one | 287.2049 | 287.2011 | 0.0038 | 1.6217 |
| 1-Deoxy Balfourodinium | 289.1618 | 289.1678 | −0.0059 | 3.1754 |
| 4-Amino-4,6-dideoxy-3-C- | 290.1687 | 290.1603 | 0.0083 | 1.2743 |
| methylmannose Me glycoside, N- | | | | |
| Me, N,2-di-Ac | | | | |
| 1-Octen-3-yl glucoside | 291.1876 | 291.1807 | 0.0069 | 1.8197 |
| 6-Hydroxy-7,9-octadecadiynoic acid | 293.2147 | 293.2116 | 0.0031 | 5.13 |
| hydroxyoctadecatrienoic acid | 295.2310 | 295.2273 | 0.0037 | 28.5706 |
| 2,5-Epoxy-6,10,14-trimethyl-9,13- | 297.2454 | 297.2429 | 0.0024 | 44.5128 |
| pentadecadiene-2,6-diol | | | | |
| 6-Isocassine | 298.2688 | 298.2746 | −0.0058 | 20.3949 |
| hydroxyoctadecenoic acid | 299.2676 | 299.2586 | 0.0090 | 15.3735 |
| 6-Isocarnavaline | 300.2883 | 300.2902 | −0.0019 | 9.6341 |
| Aplysiapyranoid D | 300.9965 | 300.9961 | 0.0004 | 0.0155 |
| lauric acid, 2-butoxyethyl ester | 301.2691 | 301.2742 | −0.0051 | 3.4098 |
| Benzastatin F | 303.2158 | 303.2072 | 0.0085 | 2.3434 |
| Acetylacrifoline | 304.1960 | 304.1912 | 0.0048 | 1.1545 |
| 8-shogaol | 305.2137 | 305.2116 | 0.0021 | 1.7856 |
| capsaicin | 306.2105 | 306.2069 | 0.0036 | 1.3473 |
| 10-paradol | 307.2209 | 307.2273 | −0.0064 | 3.3002 |
| Isonitrarine | 308.2187 | 308.2126 | 0.0061 | 1.2706 |
| linoleic acid ethylester | 309.2757 | 309.2793 | −0.0036 | 4.7311 |
| epoxyhydroxyoctadecanoic acid | 313.2726 | 313.2732 | −0.0007 | 12.6711 |
| Prosophylline | 314.2738 | 314.2695 | 0.0043 | 6.2872 |
| Batzellaside B | 318.2669 | 318.2644 | 0.0025 | 2.157 |
| galanolactone/aframodial/galanal/steviol/ | 319.2258 | 319.2273 | −0.0015 | 2.214 |
| andrograpanin | | | | |
| homocapsaicin | 320.2235 | 320.2225 | 0.0009 | 0.8313 |
| 13-Propanoyloxylupanine. | 321.2191 | 321.2178 | 0.0013 | 1.1709 |
| 3-Farnesylindole | 322.2503 | 322.2534 | −0.0031 | 1.1982 |
| Batzellaside A | 332.2871 | 332.2801 | 0.0070 | 3.0361 |
| Istamycin A | 333.2541 | 333.2502 | 0.0039 | 3.0035 |
| Fasicularine | 335.2556 | 335.2521 | 0.0036 | 2.8956 |
| pregnanetriol | 337.2808 | 337.2742 | 0.0066 | 15.2164 |
| Oxiranemethanol | 341.3028 | 341.3055 | −0.0028 | 5.3352 |
| 5,8,11,14-Eicosatetraenoic acid; 2- | 348.2992 | 348.2902 | 0.0090 | 1.8303 |
| Aminoethyl ester | | | | |
| Bahiensol | 349.3053 | 349.2954 | 0.0100 | 3.6024 |
| Plakortide H | 355.2851 | 355.2848 | 0.0003 | 28.1935 |
| 4,6-Diethyl-6-(2-ethyl-4- | 357.3026 | 357.3005 | 0.0022 | 34.5601 |
| methyloctyl)-1,2-dioxane-3-acetic | | | | |
| acid | | | | |
| 12-shoagol | 360.2706 | 360.2750 | −0.0043 | 4.3478 |
| 7,8-Epoxy-7,8-seco-8,11,13- | 361.2805 | 361.2742 | 0.0063 | 18.6669 |
| totaratriene-7,13-diol | | | | |
| 13-Epiyosgadensonol | 363.2977 | 363.2899 | 0.0079 | 2.2151 |
| Dihydroallomurolic acid | 371.2757 | 371.2797 | −0.0040 | 1.2447 |
| Emericolin B | 373.3041 | 373.3106 | −0.0065 | 3.692 |
| Ergosta-7,22-diene | 383.3702 | 383.3678 | 0.0025 | 29.0085 |
| cholestenone/cholecalciferol/dehydro | 385.3500 | 385.3470 | 0.0030 | 4.131 |
| cholesterol | | | | |
| Mycestericin G | 388.3161 | 388.3063 | 0.0098 | 3.4202 |
| 16,25-Epidioxy-17(24)-scalaren-6-ol | 389.3071 | 389.3055 | 0.0016 | 3.1682 |
| Edulimide | 393.2262 | 393.2178 | 0.0084 | 0.8537 |
| 24-Nor-18a-olean-12-ene | 397.3835 | 397.3834 | 0.0001 | 57.9824 |
| solanine D | 400.3488 | 400.3579 | −0.0091 | 5.0376 |
| 12-Hydroxy-24-methyl-24-oxo-16- | 401.3123 | 401.3055 | 0.0067 | 6.8373 |
| scalaren-25-al | | | | |
| Spectamine A | 402.3043 | 402.3008 | 0.0035 | 3.2478 |
| 5-(3,13-Eicosadienyl)-2-furanacetic | 403.3196 | 403.3212 | −0.0016 | 3.4334 |
| acid | | | | |
| Baleabuxidine I | 405.3170 | 405.3117 | 0.0054 | 2.5441 |
| 2,6,10,15,19,23-Hexamethyl- | 409.3850 | 409.3834 | 0.0016 | 28.1655 |
| 2,6,10,12,14,18,22-tetracosaheptaene | | | | |
| 12,21-Baccharadiene | 411.3912 | 411.3991 | −0.0078 | 10.5027 |
| fucosterol/sitosterone/spinasterol/stig | 413.3827 | 413.3783 | 0.0044 | 6.5208 |
| masterol/sitostenone/chondrillasterol | | | | |
| 24,28-Dihydro-15-azasterol | 414.3778 | 414.3736 | 0.0043 | 4.9474 |
| 8,9-Epoxy-8,9-secoergosta-7,9(11)- | 415.3618 | 415.3576 | 0.0042 | 3.6915 |
| dien-3-ol | | | | |
| tomatidine | 416.3518 | 416.3528 | −0.0010 | 2.9017 |
| Buxidienine F | 417.3474 | 417.3481 | −0.0007 | 2.6687 |
| amyrenone/lupenone | 425.3832 | 425.3783 | 0.0049 | 12.2791 |
| cholesteryl acetate | 429.3806 | 429.3732 | 0.0074 | 8.2703 |
| Edpetilidinine | 430.3749 | 430.3685 | 0.0064 | 3.7021 |
| 9,11-Epoxycholest-7-ene-3,5,6-triol | 433.3339 | 433.3318 | 0.0021 | 3.0062 |
| Cholest-5-ene-3,16,22,26-tetrol | 435.3436 | 435.3474 | −0.0038 | 4.317 |
| Ergosta-4,6,8(14),22-tetraen-3-ylurea | 437.3631 | 437.3532 | 0.0099 | 3.4071 |
| Nb-Nonadecanoyltryptamine | 441.3933 | 441.3845 | 0.0088 | 11.9182 |
| 21-O-Phosphate, Hydrocortisone | 443.1907 | 443.1835 | 0.0072 | 0.0229 |
| phosphate | | | | |
| 12-Oleanene-3,22-diol | 443.3873 | 443.3889 | −0.0016 | 5.791 |
| 5,6-Epoxystigmast-8(14)-ene-3,7- | 445.3720 | 445.3681 | 0.0038 | 6.4695 |
| diol | | | | |
| 3-Deamino-3-hydroxysolanocapsine | 446.3665 | 446.3634 | 0.0031 | 4.3241 |
| 6-Deoxodolichosterone | 449.3583 | 449.3631 | −0.0048 | 1.6646 |
| vitamin K1(phytonadione) | 451.3620 | 451.3576 | 0.0044 | 2.0443 |
| 22,25-Epoxystigmast-7-ene-3,16,26- | 461.3723 | 461.3631 | 0.0093 | 2.3853 |
| triol | | | | |
| 30-Epibatzelladine D | 463.3813 | 463.3760 | 0.0053 | 3.6191 |
| 3-Epipachysamine H | 465.3913 | 465.3845 | 0.0068 | 2.9902 |
| Stellettasterol | 469.3541 | 469.3529 | 0.0012 | 1.0517 |
| soyasapogenol A | 474.3767 | 474.3709 | 0.0058 | 1.6654 |
| 3,24,25-Trihydroxycucurbit-5-en- | 475.3799 | 475.3787 | 0.0011 | 2.0162 |
| 11-one | | | | |
| 21-Baccharene-3,18,23,28-tetrol | 477.3889 | 477.3944 | −0.0054 | 2.0492 |
| Efrapeptin B | 479.3853 | 479.3835 | 0.0018 | 3.0083 |
| Pachysanaximine A | 481.3851 | 481.3794 | 0.0058 | 2.3431 |
| Ergostane-1,3,5,6,18,25-hexol | 483.3609 | 483.3685 | −0.0077 | 1.1699 |
| Batzelladine E | 487.3669 | 487.3760 | −0.0091 | 1.1878 |
| Batzelladine C | 489.3841 | 489.3917 | −0.0076 | 1.2632 |
| Emindole PA | 490.3764 | 490.3685 | 0.0079 | 0.6128 |
| cholesteryl benzoate | 491.3950 | 491.3889 | 0.0061 | 2.603 |
| 21,28-Epoxy-3,18,23,29- | 493.3948 | 493.3893 | 0.0055 | 3.5156 |
| baccharanetetrol | | | | |
| acetyl-boswellic acid | 497.3999 | 497.3994 | 0.0005 | 1.0476 |
| Isobutyrylbaleabuxidine F | 503.3799 | 503.3849 | −0.0049 | 1.534 |
| N-Isobutyrylbaleabuxaline F | 505.4091 | 505.4005 | 0.0086 | 2.6974 |
| 14- | 509.4041 | 509.3994 | 0.0046 | 1.167 |
| Octadecyloxydehydrocacalohastine | | | | |
| Stearoylplorantinone B | 517.4238 | 517.4257 | −0.0018 | 0.9833 |
| betulin diacetate | 527.4158 | 527.4100 | 0.0058 | 0.7099 |
| Buxhejramine | 529.4395 | 529.4369 | 0.0026 | 0.7973 |
| 29-(2,3,4,5- | 547.4804 | 547.4726 | 0.0078 | 1.8351 |
| Tetrahydroxypentyl)hopane | | | | |
| 12-Cinnamoyl, 11-Ac, | 553.2889 | 553.2801 | 0.0088 | 0.0638 |
| Condurangogenin E | | | | |
| tricaprin | 555.4555 | 555.4624 | −0.0069 | 0.656 |
| 35-Me ether-(2,3,4,5- | 559.4785 | 559.4726 | 0.0059 | 1.5321 |
| Tetrahydroxypentyl)-6-hopene | | | | |
| Lactone dimer. 13,26-Dihexyl-1,14- | 561.4951 | 561.4883 | 0.0068 | 5.6426 |
| dioxacyclohexacosa-10,23-diene- | | | | |
| 2,15-dione | | | | |
| Nb-Octacosanoyltryptamine | 567.5281 | 567.5253 | 0.0028 | 1.1219 |
| Heptacosyl (E)-ferulate | 573.4850 | 573.4883 | −0.0032 | 1.3599 |
| 5,8,11,14,17-Eicosapentaenoyl | 581.5259 | 581.5297 | −0.0038 | 3.1448 |
| 4,5α:24R,25-Diepoxide, 3-octanoyl | 587.4990 | 587.5039 | −0.0049 | 0.4913 |
| Reticulatain 2 | 593.5073 | 593.5145 | −0.0072 | 2.6179 |
| 10,18-Epoxy-1(19),7,11,13- | 599.5050 | 599.5039 | 0.0011 | 9.1365 |
| xenicatetraene-6,17-diol | | | | |
| Glycerol 1-(9E-octadecenoate) 3- | 621.5405 | 621.5458 | −0.0052 | 1.794 |
| (9Z-octadecenoate) | | | | |
| mogroside V-4glc | 639.4515 | 639.4472 | 0.0043 | 0.0358 |
| trilaurin | 639.5566 | 639.5563 | 0.0003 | 0.4117 |
| Diosgenin palmitate | 653.5548 | 653.5509 | 0.0040 | 1.181 |
| 18-Eicosanoyl, 1-Ac | 659.5675 | 659.5614 | 0.0060 | 0.5374 |
| 11,12-Epoxy-14-taraxeren-3- | 679.6128 | 679.6029 | 0.0099 | 0.7229 |
| ol; Hexadecanoyl | | | | |
| 3-O-Pentadecanoyl | 681.5910 | 681.5822 | 0.0088 | 0.5123 |
| Manzamenone B | 743.5889 | 743.5826 | 0.0064 | 0.7322 |
| Ergost-5-en-3-ol; O-(6-O-9Z- | 827.6802 | 827.6765 | 0.0038 | 0.5826 |
| Octadecenoyl-b-D-glucopyranoside) | | | | |
| 16-Acetyl, 21-O-(3,4-diangeloyl--D- | 859.5171 | 859.5207 | −0.0037 | 0.1042 |
| fucopyranoside)-12-Oleanene- | | | | |
| 3,16,21,22,24,28-hexol | | | | |
| Thermozeaxanthin 17 | 983.7312 | 983.7340 | −0.0028 | 0.3615 |

D. COX-1 and COX-2 Selective and Non-Selective Inhibition

All reagents and solutions were prepared according to the protocols established by Cayman Chemicals (Ann Arbor, Mich.) for the COX-1 and COX-2 inhibition assays. Two procedures were utilized to assess the COX-1/COX-2-specific and non-specific activities.

Prostaglandin Production Inhibition: Extracts were diluted in dimethylsulfoxide (DMSO), and then diluted in reaction buffer so that the final concentration of DMSO was 1%. Reactions were either run with COX-1 or COX-2 in the presence of Heme. Wells containing potential inhibitors (SRB extracts), non-inhibitor (100% activity) or background wells (heat inactivated enzyme) along with appropriate blanks were prepared. Solutions were placed in a 37° C. incubator for 15 minutes prior to running the reaction. Arachidonic acid was added and mixed and the reaction proceeded for 2 minutes. The reaction was stopped by addition of 1 M HCl to each well, then reducing the Prostaglandin H₂ product to ProstaglandinG F₂, which was quantified using EIA.

Quantification of Prostaglandin with EIA: The EIA assay plate was provided in the Cayman Chemicals screening kit. Aliquots, 50 μL, of the reaction products (PGF₂) from prostaglandin production were added to their respective wells. Total activity and blank wells received 150 μL of EIA buffer, non-specific binding wells received 100 μL of EIA buffer, and maximum binding wells received 50 μL of EIA buffer. COX 100% activity wells, non-specific binding, background, maximum binding, standards, and extract wells received 50 μL of tracer. COX 100% activity, background, maximum binding, standards, and extract wells all received 50 μL of antiserum. Reaction in EIA plates was allowed to run for 18 hours at room temperature. Plates were washed with wash buffer and then 200 μL Ellman's Reagent was added to all wells and 5 μL tracer was added to total activity well. The color development was quantified at 409 nm in a Tecan M200 microplate reader.

The IC₅₀ values for COX-1 inhibition by SRB extract 1 and SRB extract 2 are 305 μg mL⁻¹ and 310 μg mL⁻¹, respectively based on triplicate experiments (Table 4). The IC₅₀ values for COX-2 inhibition by SRB extract 1 and SRB extract 2 are 29 μg mL⁻¹ and 19 μg mL⁻¹, respectively based on triplicate experiments (Table 4).

E. 5-Lipoxygenase Inhibition

The 5-lipoxygenase (5-LOX) activity was determined by monitoring leukotriene formation using purified 5-LOX according to the manufacturer's protocol (Cayman Chemical, Ann Arbor Mich.). In a 96-well format, 90 μL of 5-LOX was added to 10 μL of extract, followed by 10 μL of arachidonic acid and shaken for 5 minutes at 25° C. After shaking, 100 μL of Chromagen developing reagent was added to each well and the plate was again shaken for 5 minutes. Absorbance at 500 nm was measured in each well using a Tecan M200 microplate reader. The IC₅₀ value was determined to be 396 μg mL⁻¹, based on triplicate experiments (Table 4). The COX-2 to 5-LOX inhibition ratio for SRB Extract 2 is ca. 21:1.

F. COX and LOX Inhibition of SRB extract 3

The IC₅₀ value for inhibition of COX-1 by SRB extract 3 is 47.9 μg mL⁻¹, for COX-2 is 11.42 μg mL⁻¹, and for 5-LOX is 197.3 μg mL⁻¹ based on triplicate experiments (Table 4). SRB Extract 3 is enriched in COX and LOX inhibition activities with a COX-2 to 5-LOX inhibition activity ratio of ca. 18:1. SRB extract 3 reveals some additive or perhaps synergistic effects when combining SRB Extract 1 and SRB Extract 2 in a ratio of 1:6 as the IC₅₀ values, notably for COX-1 inhibition, are reduced nearly 7-fold, while the IC₅₀ values for COX-2 and 5-LOX inhibition are reduced by ca. 2-fold.

TABLE 4
Summary of the IC₅₀ values of SRB Extract 1, SRB Extract 2 and SRB
Extract 3 against COX-1, COX-2 and 5-LOX enzymes.

| | | | | Extract 2 | | | Extract 3 | | |
|---|---|---|---|---|---|---|---|---|---|
| | Extract 1 | | | IC₅₀ | | | IC₅₀ | | |
| Enzyme | IC₅₀ (μg mL⁻¹) | R² | N | (μg mL⁻¹) | R² | N | (μg mL⁻¹) | R² | N |
| COX-1 | 305 | 0.97 | 15 | 310 | 0.93 | 15 | 48 | 0.86 | 15 |
| COX-2 | 29 | 0.91 | 21 | 19 | 0.92 | 24 | 11 | 0.85 | 24 |
| 5-LOX | ND | ND | ND | 396 | 0.95 | 18 | 197 | 0.95 | 24 |

K. Assessment of Cellular toxicity

Cellular toxicity of SRB extract 1 against 293HEK cells was determined using an MTT assay. Briefly, monolayers of 293HEK cells were prepared in a 96-well plate format, and incubated for 16-24 hours to allow the monolayer to form. After the monolayer has formed, the 293HEK cells are incubated in the presence or absence of varying concentrations of SRB extract 1 for 16-24 hours. The MTT imaging reagent was added to all wells containing a monolayer and incubated for an additional 3-4 hours. The media was removed and 100 μL crystal dissolving agent was added to all wells. The plate was read at 570 nm using a Biotek Synergy 4 plate reader.

The percentage of living 293HEK cells in the extract containing wells is determined based on comparison to the control wells (no extract). The cytotoxicity concentration (CC₅₀) is determined from the percentage of living cells in the extract containing wells and the control wells. The CC₅₀ for SRB extract 1 is greater than 1000 μg mL⁻¹. When the CC₅₀ is known, the Selectivity Index (SI; CC₅₀/IC₅₀) can be determined for each endpoint. The SI is a measure of extract activity on the enzyme/endpoint vs. direct activity on cells. An SI>1 indicates an active extract, and an SI>10 indicates a highly active extract. The SI's for SRB Extract 1 against COX-1 and COX-2 are >3 and >34 respectively, indicating that the inhibitory activity against COX-1 and COX-2 from SRB extract 1 will not cause toxicity to cells.

L. Summary of Bioactives

The known compounds in SRB Extract 1 (COX) are summarized with their molecular mass, chemical class, relative abundance, and weight per 100 mg dose (based on their relative abundances) in Table 5. Among the 9 known bioactives in SRB Extract 1, only one compound, 12-Shogaol, a gingerol, was previously reported to possess anti-inflammatory activities. Of the known compounds, conyrin and epiloliode, both alkaloids, and nonanedoic acid, a fatty acid, have strong of COX-2 inhibition. The COX-2 inhibition activities of these compounds have not previously been reported.

TABLE 5
Summary of active compounds identified in SRB Extract 1.

| | | | | Relative | wt per |
|---|---|---|---|---|---|
| | Molecular | Molecular | Chemical | Abundance | 100 mg |
| Compound Name | Formula | Mass | Class | (%) | (μg) |
| Valeric acid/ | C₅H₁₀O₂ + H⁺ | 102.07 | fatty acid | 1.8 | 34 |
| methylbutyric acid | | | | | |
| norcamphor/heptadienal | C₇H₁₀O + H⁺ | 110.08 | terpene | 7.61 | 145 |
| conyrin | C₈H₁₁N + H⁺ | 121.10 | alkaloid | 1.95 | 37 |
| ocimene/camphene/ | C₁₀H₁₆ + H⁺ | 136.12 | terpene | 19.65 | 374 |
| adamantane | | | | | |
| lysine | C₆H₁₄N₂O₂ + H⁺ | 146.12 | amino acid | 7.96 | 152 |
| carvacrol/thymol/ | C₁₀H₁₄O + H⁺ | 150.12 | terpenol | 21.1 | 402 |
| cymenol | | | | | |
| Nonanedioic acid anhydride | C₉H₁₄O₃ + H⁺ | 170.11 | fatty acid | 4.52 | 86 |
| Epiloliolide | C₁₁H₁₆O₃ + H⁺ | 196.12 | alkaloid | 19.92 | 379 |
| 12-shogaol | C₂₃H₃₆O₃ + H⁺ | 360.28 | gingerol | 4.17 | 79 |

In Table 6, the known compounds in SRB Extract 2 are summarized with their molecular mass, chemical class, relative abundance, and weight per 100-mg dose (based on their abundances). These compounds have no literature reported anti-inflammatory activities; therefore, the 5-LOX inhibition activity of these compounds described here is novel.

TABLE 6
Summary of active compounds identified in SRB Extract 2.

| | | | | Relative | Wt per |
|---|---|---|---|---|---|
| | Molecular | Molecular | Chemical | Abundance | 100 mg |
| Compound Name | Formula | Mass | Class | (%) | (μg) |
| 6-methyl-5-hepten-2-one | C₈H₁₄O + H⁺ | 126.11 | terpene | 4.09 | 321 |
| histidinol | C₆H₁₁N₃O + H⁺ | 142.11 | imidazole | 11.25 | 883 |
| 2,6-tropanediol | C₈H₁₅NO₂ + H⁺ | 157.21 | alkaloid | 2.90 | 228 |
| tryptamine | C₁₀H₁₂N₂ + H⁺ | 160.12 | amino acid | 1.60 | 126 |
| 2,4-hexadienoic acid | C₁₀H₁₇NO + H⁺ | 167.25 | fatty acid | 0.41 | 32 |
| isobutylamide | | | | | |
| Acetylaburnine | C₁₀H₁₇NO₂ + H⁺ | 183.13 | alkaloid | 1.20 | 94 |
| Nonanedioic acid diamide | C₉H₁₈N₂O₂ + H⁺ | 187.14 | fatty acid | 0.99 | 78 |
| curcumene | C₁₅H₂₂ + H⁺ | 202.18 | terpene | 2.29 | 179 |
| Farnesatrienetriol | C₁₅H₂₆O₃ + H⁺ | 254.36 | terpene | 5.05 | 397 |
| Farnesylacetone | C₁₈H₃₀O + H⁺ | 262.43 | terpene | 19.96 | 1,568 |
| Octadecatrienol | C₁₈H₃₂O + H⁺ | 264.45 | fatty acid | 10.31 | 809 |
| octadecatrienoic acid | C₁₈H₃₀O₂ + H⁺ | 278.43 | fatty acid | 50.74 | 3,984 |
| hydroxyoctadecatrienoic acid | C₁₈H₃₀O₃ + H⁺ | 294.43 | fatty acid | 11.50 | 903 |
| hydroxyoctadecenoic acid | C₁₈H₃₄O₃ + H⁺ | 298.46 | fatty acid | 13.73 | 1,078 |
| epoxyhydroxyoctadecanoic | C₁₈H₃₂O₄ + H⁺ | 312.27 | fatty acid | 5.49 | 431 |
| acid | | | | | |

In Table 7, the known active compounds in SRB extract 3 are summarized with their molecular mass, chemical class, relative abundances, and weight per 100 mg dose. These compounds have no literature reported anti-inflammatory activities except 12-shogaol. Therefore, the COX and 5-LOX inhibition activity of the other compounds described here are novel.

TABLE 7
Summary of active compounds identified in SRB Extract 3.

| | | | | Relative | Wt per |
|---|---|---|---|---|---|
| | Molecular | Molecular | Chemical | Abundance | 100 mg |
| Compound Name | Formula | Mass | Class | (%) | (μg) |
| norcamphor/heptadienal | C₇H₁₀O + H⁺ | 110.08 | terpene | 0.45 | 18 |
| 6-methyl-5-hepten-2-one | C₈H₁₄O + H⁺ | 126.11 | terpene | 3.24 | 127 |
| ocimene/camphene/ | C₁₀H₁₆ + H⁺ | 136.12 | terpene | 1.42 | 56 |
| adamantane | | | | | |
| histidinol | C₆H₁₁N₃O + H⁺ | 142.11 | imidazole | 2.86 | 112 |
| lysine | C₆H₁₄N₂O₂ + H⁺ | 146.12 | amino acid | 0.42 | 17 |
| tryptamine | C₁₀H₁₂N₂ + H⁺ | 160.12 | amino acid | 0.39 | 15 |
| Nonanedioic acid | C₉H₁₄O₃ + H⁺ | 170.11 | fatty acid | 1.81 | 71 |
| anhydride | | | | | |
| Nonanedioic acid diamide | C₉H₁₈N₂O₂ + H⁺ | 187.14 | fatty acid | 1.92 | 75 |
| Epiloliolide | C₁₁H₁₆O₃ + H⁺ | 196.12 | alkaloid | 5.74 | 226 |
| Farnesatrienetriol | C₁₅H₂₆O₃ + H⁺ | 254.36 | terpene | 5.08 | 199 |
| Farnesylacetone | C₁₈H₃₀O + H⁺ | 262.43 | terpene | 27.13 | 1066 |
| Octadecatrienol | C₁₈H₃₂O + H⁺ | 264.45 | fatty acid | 16.61 | 653 |
| octadecatrienoic acid | C₁₈H₃₀O₂ + H⁺ | 278.43 | fatty acid | 100.00 | 3928 |
| hydroxyoctadecatrienoic | C₁₈H₃₀O₃ + H⁺ | 294.43 | fatty acid | 28.57 | 1122 |
| acid | | | | | |
| hydroxyoctadecenoic acid | C₁₈H₃₄O₃ + H⁺ | 298.46 | fatty acid | 15.37 | 604 |
| epoxyhydroxyoctadecanoic | C₁₈H₃₂O₄ + H⁺ | 312.27 | fatty acid | 12.67 | 498 |
| acid | | | | | |
| 12-shogaol | C₂₃H₃₆O₃ + H⁺ | 360.28 | gingerol | 18.67 | 733 |

M. Human Pharmacokinetics of SRB Anti-Inflammatory Bioactive Compounds

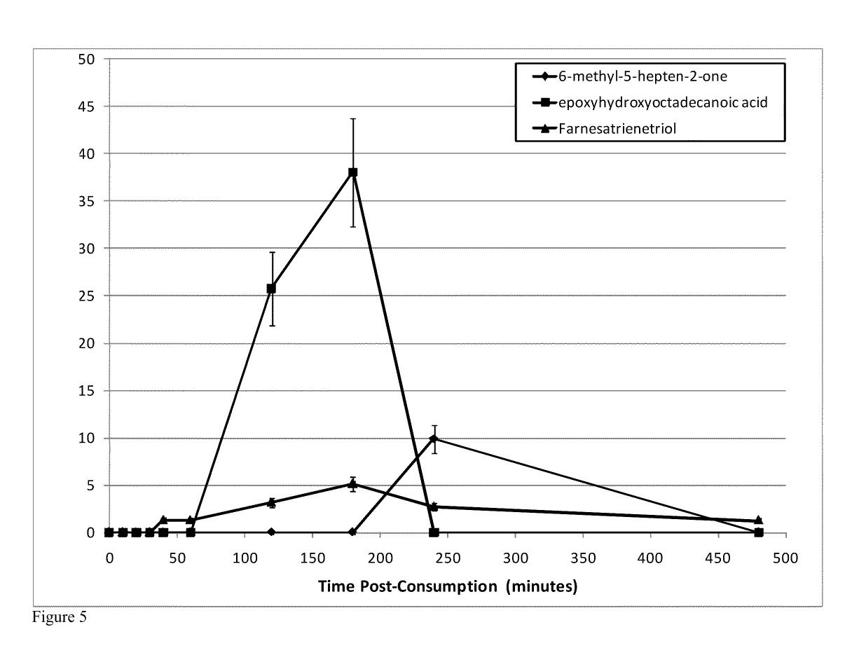

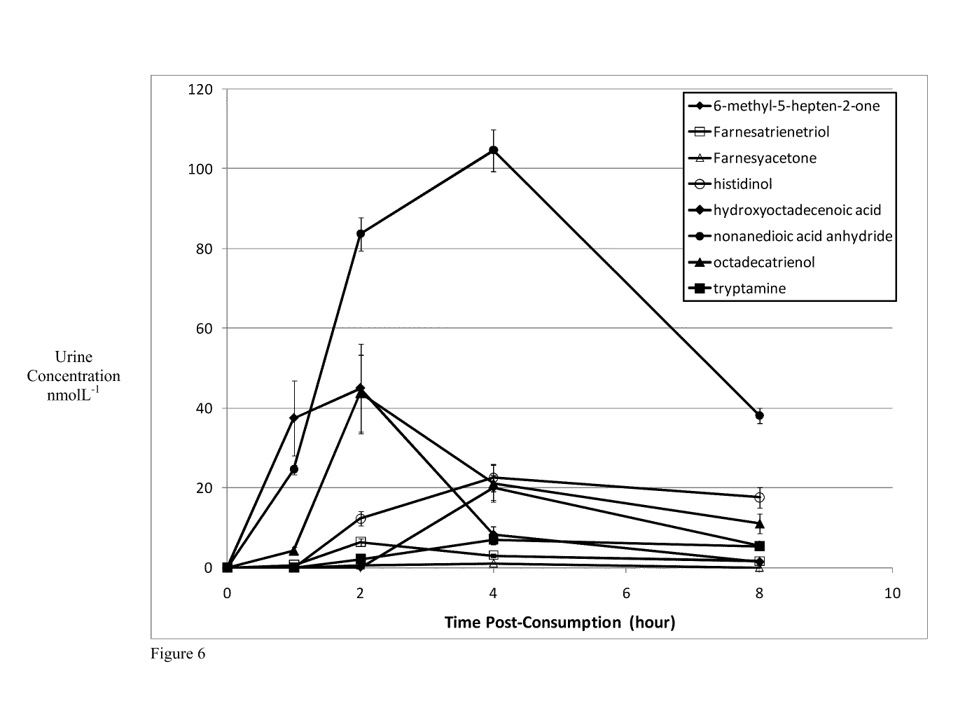

Five healthy consenting adults ranging in age from 25 to 50 were instructed not to consume foods rich in polyphenolics 24 hr prior to the initiation of the study. A certified individual collected blood samples at several time intervals between 0 and 480 minutes after two vegcaps containing a total of 180-mg of SRB Extract 3 were ingested. Immediately after the time zero time point, blood samples were collected two vegcaps containing a total of 180-mg of SRB Extract 3 were administered. Blood samples were handled with approved protocols and precautions, centrifuged to remove cells and the serum fraction was collected and frozen. Blood was not treated with heparin to avoid any analytical interference. Serum samples were stored frozen until analysis. The serum was extracted with an equal volume of neat ethanol (USP) to minimize background of proteins, peptides, and polysaccharides present in serum. The ethanol extract was centrifuged for 10 minutes at 4° C., the supernatant was removed, concentrated to 200 μL volume and DART TOF-MS analyses was conducted as described above to identify the bioactive components of SRB Extract 3 that are taken up into the blood between 45 and 240 minutes and excreted in the urine. FIGS. 5 and 6 provide the human pharmacokinetic profile of the bioavailable SRB bioactives in serum and urine respectively.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A stabilized rice bran extract comprising at least one compound selected from the group consisting of 0.01 to 10% by weight valeric/methylbutyric acid, 0.01 to 10% by weight of norcamphor/heptadienal, 0.01 to 10% by weight conyrin, 0.05 to 10% by weight ocimene/camphene/adamantane 0.01 to 10% by weight lysine, 0.05 to 10% by weight carvacrol/thymol/cymenol, 0.01 to 10% by weight nonanedioic acid anhydride, 0.05 to 10% by weight epiloliolide, and 0.01 to 10% by weight of 12-shogoal.

2. The stabilized rice bran extract of claim 1, comprising at least one compound selected from the group consisting of 0.01 to 2% by weight valeric/methylbutyric acid, 0.05 to 3% by weight of norcamphor/heptadienal, 0.01 to 2% by weight conyrin, 0.05 to 3% by weight ocimene/camphene/adamantane 0.05 to 3% by weight lysine, 0.1 to 5% by weight carvacrol/thymol/cymenol, 0.01 to 2% by weight nonanedioic acid anhydride, 0.1 to 5% by weight epiloliolide, and 0.01 to 2% by weight of 12-shogaol.

3. A stabilized rice bran extract comprising at least one compound selected from the group consisting of 5 to 300 μg valeric/methylbutyric acid, 50 to 500 μg norcamphor/heptadienal, 5 to 300 μg conyrin, 100 to 1,000 μg ocimene/camphene/adamantane, 50 to 500 μg lysine, 100 to 1,000 μg carvacrol/thymol/cymenol, 10 to 500 μg nonanedioic acid anhydride, 100 to 1000 μg epiloliolide, and 5 to 500 μg 12-shogaol, per 100 mg of the extract.

4. A stabilized rice bran extract comprising carvacrol/thymol/cymenol, 5 to 30% valeric/methylbutyric acid by weight of the carvacrol/thymol/cymenol, 10 to 50% norcamphor/heptadienal by weight of the carvacrol/thymol/cymenol, 1 to 20% conyrin by weight of the carvacrol/thymol/cymenol, 75 to 125% ocimene/camphene/adamantine by weight of the carvacrol/thymol/cymenol, 10 to 50% lysine by weight of the carvacrol/thymol/cymenol, 5 to 50% nonanedioic acid anhydride, 75 to 125% epiloliolide by weight of the carvacrol/thymol/cymenol, and 5 to 50% 12-shogaol by weight of the carvacrol/thymol/cymenol.

5. A stabilized rice bran extract comprising at least one compound selected from the group consisting of 0.05 to 10% 6-methyl-5-hepten-2-one, 0.1 to 10% histidinol, 0.05 to 10% 2,6-tropanediol, 0.05 to 10% tryptamine, 0.01 to 5% 2,4-hexanienoic acid isobutylamide, 0.01 to 5% acetylaburnine, 0.01 to 5% nonanedioic acid diamide, 0.05 to 10% curcumene, 0.05 to 10% famesatrienetriol, 0.1 to 20% farnesylacetone, 0.1 to 10% octadecatrienol, 0.5 to 20% octadecatrienoic acid, 0.1 to 10% hydroxyoctadecatrienoic acid, 0.1 to 20% hydroxyoctadecenoic acid, and 0.1 to 10% epoxyhydroxyoctadecanoic acid.

6. The stabilized rice bran extract of claim 5, comprising at least one compound selected from the group consisting of 0.05 to 2% 6-methyl-5-hepten-2-one, 0.1 to 2% histidinol, 0.05 to 2% 2,6-tropanediol, 0.05 to 2% tryptamine, 0.01 to 1% 2,4-hexanienoic acid isobutylamide, 0.01 to 3% acetylaburnine, 0.01 to 2% nonanedioic acid diamide, 0.05 to 2% curcumene, 0.1 to 2% farnesatrienetriol, 0.5 to 5% farnesylacetone, 0.1 to 2% octadecatrienol, 1 to 10% octadecatrienoic acid, 0.1 to 2% hydroxyoctadecatrienoic acid, 0.5 to 5% hydroxyoctadecenoic acid, and 0.1 to 2% epoxyhydroxyoctadecanoic acid.

7. A stabilized rice bran extract comprising at least one compound selected from 25 to 1000 μg 6-methyl-5-hepten-2-one, 100 to 2000 μg histidinol, 25 to 500 μg 2,6-tropanediol, 10 to 500 μg tryptamine, 5 to 100 μg 2,4-hexanienoic acid isobutylamide, 10 to 500 μg acetylaburnine, 10 to 500 μg nonanedioic acid diamide, 25 to 500 μg curcumene, 50 to 1000 farnesatrienetriol, 500 to 5000 μg farnesylacetone, 100 to 2000 μg octadecatrienol, 500 to 10,000 μg octadecatrienoic acid, 100 to 2000 μg hydroxyoctadecatrienoic acid, 100 to 2000 pg hydroxyoctadecenoic acid, and 50 to 2000 μg epoxyhydroxyoctadecanoic acid.

8. A stabilized rice bran extract comprising octadecatrienoic acid, 1 to 20% 6-methyl-5-hepten-2-one by weight of the octadecatrienoic acid, 5 to 50% histidinol by weight of the octadecatrienoic acid, 1 to 20% 2,6-tropanediol by weight of the octadecatrienoic acid, 0.5 to 15% tryptamine by weight of the octadecatrienoic acid, 0.1 to 5% 2,4-hexanienoic acid isobutylamide by weight of the octadecatrienoic acid, 0.5 to 10% acetylaburnine by weight of the octadecatrienoic acid, 0.5 to 10% nonanedioic acid diamide by weight of the octadecatrienoic acid, 1 to 15% curcumene by weight of the octadecatrienoic acid, 1 to 25% famesatrienetriol by weight of the octadecatrienoic acid, 10 to 75% farnesylacetone by weight of the octadecatrienoic acid, 5 to 50% octadecatrienol by weight of the octadecatrienoic acid, 5 to 50% hydroxyoctadecatrienoic acid by weight of the octadecatrienoic acid, 5 to 50% hydroxyoctadecenoic acid by weight of the octadecatrienoic acid, and 1 to 20% epoxyhydroxyoctadecanoic acid by weight of the octadecatrienoic acid.

9. A stabilized rice bran extract comprising at least one compound selected from the group consisting of 0.001 to 5% norcamphor/heptadienal, 0.05 to 5% 6-methyl-5-hepten-2-one, 0.001 to 5% ocimene/camphene/adamantane 0.05 to 5% histidinol, 0.001 to 5% lysine, 0.001 to 5% tryptamine, 0.05 to 5% nonanedioic acid anhydride, 0.05 to 5% nonanedioic acid diamide, 0.05 to 5% epiloliolide, 0.05 to 5% farnesatrienetriol, 0.1 to 10% farnesylacetone, 0.1 to 10% octadecatrienol, 1 to 10% octadecatrienoic acid, 0.1 to 10% hydroxyoctadecatrienoic acid, 0.1 to 5% hydroxyoctadecenoic acid, 0.1 to 5% epoxyhydroxyoctadecanoic acid, and 0.1 to 5% 12-shogaol.

10. The stabilized rice bran extract of claim 9 comprising at least one compound selected from the group consisting of 0.001 to 1% norcamphor/heptadienal, 0.05 to 1% 6-methyl-5-hepten-2-one, 0.001 to 1% ocimene/camphene/adamantane 0.05 to 1% histidinol, 0.001 to 1% lysine, 0.001 to 1% tryptamine, 0.05 to 1% nonanedioic acid anhydride, 0.05 to 1% nonanedioic acid diamide, 0.05 to 1% epiloliolide, 0.05 to 1% farnesatrienetriol, 0.5 to 2% farnesylacetone, 0.1 to 1% octadecatrienol, 1 to 5% octadecatrienoic acid, 0.5 to 2% hydroxyoctadecatrienoic acid, 0.1 to 1% hydroxyoctadecenoic acid, 0.1 to 1% epoxyhydroxyoctadecanoic acid, and 0.1 to 1.5% 12-shogaol.

11. A stabilized rice bran extract comprising at least one compound selected from the group consisting of 5 to 100 μg norcamphor/heptadienal, 10 to 500 μg 6-methyl-5-hepten-2-one, 5 to 100 μg ocimene/camphene/adamantane 10 to 500 μg histidinol, 5 to 100 μg lysine, 5 to 100 μg tryptamine, 100 to 500 pg nonanedioic acid anhydride, 10 to 100 μg nonanedioic acid diamide, 50 to 1000 μg epiloliolide, 10 to 1000 μg farnesatrienetriol, 100 to 5000 μg farnesylacetone, 50 to 2500 μg octadecatrienol, 500 to 10000 μg octadecatrienoic acid, 100 to 5000 μg hydroxyoctadecatrienoic acid, 100 to 2500 μg hydroxyoctadecenoic acid, 50 to 1500 μg epoxyhydroxyoctadecanoic acid, and 100 to 2500 μg 12-shogaol, per 100 mg of the extract.

12. A stabilized rice bran extract comprising octadecatrienoic acid, 0.1 to 5% norcamphor/heptadienal by weight of the octadecatrienoic acid, 0.5 to 10% 6-methyl-5-hepten-2-one by weight of the octadecatrienoic acid, 0.1 to 5% ocimene/camphene/adamantane by weight of the octadecatrienoic acid, 0.5 to 10% histidinol by weight of the octadecatrienoic acid, 0.1 to 5% lysine by weight of the octadecatrienoic acid, 0.1 to 5% tryptamine by weight of the octadecatrienoic acid, 0.1 to 10% μg nonanedioic acid anhydride by weight of the octadecatrienoic acid, 0.1 to 10% nonanedioic acid diamide by weight of the octadecatrienoic acid, 1 to 20% epiloliolide by weight of the octadecatrienoic acid, 1 to 20% famesatrienetriol by weight of the octadecatrienoic acid, 5 to 75% farnesylacetone by weight of the octadecatrienoic acid, 5 to 50% octadecatrienol by weight of the octadecatrienoic acid, 5 to 75% hydroxyoctadecatrienoic acid by weight of the octadecatrienoic acid, 5 to 50% hydroxyoctadecenoic acid by weight of the octadecatrienoic acid, 5 to 50% epoxyhydroxyoctadecanoic acid by weight of the octadecatrienoic acid, and 5 to 50% 12-shogaol by weight of the octadecatrienoic acid.

13. A stabilized rice bran extract having a fraction comprising a Direct Analysis in Real Time (DART) mass spectrometry chromatogram of any of FIGS. 2, 3, and 4.

14. The stabilized rice bran extract of claim 1, wherein the extract has an IC₅₀ value for COX-1 inhibition of less than 1000 μg/mL.

15. The stabilized rice bran extract of claim 14, wherein the IC₅₀ value for COX-1 inhibition is about 1 μg/mL to 500 μg/mL.

16. The stabilized rice bran extract of claim 15, wherein the IC₅₀ value for COX-1 inhibition is about 5 μg/mL to 400 μg/mL.

17. The stabilized rice bran extract of claim 16, wherein the IC₅₀ value for COX-1 inhibition is about 10 μg/mL to 350 μg/mL.

18. The stabilized rice bran extract of claim 1, wherein the extract has an IC₅₀ value for COX-2 inhibition is less than 1000 μg/mL.

19. The stabilized rice bran extract of claim 18, wherein the IC₅₀ value for COX-2 inhibition is about 0.5 μg/mL to 250 μg/mL.

20. The stabilized rice bran extract of claim 18, wherein the IC₅₀ value for COX-2 inhibition is about 1 μg/mL to 100 μg/mL.

21. The stabilized rice bran extract of claim 20, wherein the IC₅₀ value for COX-2 inhibition is about 5 μg/mL to 50 μg/mL.

22. The stabilized rice bran extract of claim 1, wherein the extract has an IC₅₀ value for 5-LOX inhibition of less than 1000 μg/mL.

23. The stabilized rice bran extract of claim 22, wherein the IC₅₀ for 5-LOX inhibition about 1 μg/mL to 500 μg/mL.

24. The stabilized rice bran extract of claim 23, wherein the IC₅₀ for 5-LOX inhibition about 10 μg/mL to 500 μg/mL.

25. The stabilized rice bran extract of claim 24, wherein the IC₅₀ for 5-LOX inhibition about 25 μg/mL to 400 μg/mL.

26. The stabilized rice bran extract of claim 25, wherein the IC₅₀ for 5-LOX inhibition about 50 μg/mL to 500 μg/mL.

27. A pharmaceutical composition comprising a stabilized rice bran extract of claim 1 and a pharmaceutically acceptable carrier.

28. A method of treating or preventing an inflammatory disorder in a subject comprising administering to a subject in need thereof a therapeutically effective amount of the composition of claim 27.

29. The method of claim 28, wherein the pharmaceutical composition is formulated as a lotion, cream, ointment, oil, paste or transdermal patch and the administration is topical.

30. The method of claim 28, wherein the pharmaceutical composition is formulated as a functional food, dietary supplement, powder or beverage.

31. The method of claim 28, wherein the inflammatory disorder is acute.

32. The method of claim 28, wherein the inflammatory disorder is chronic.

33. The method of claim 28, wherein the inflammatory disorder is arthritis, asthma, gout, tendonitis, bursitis, polymyalgia, rheumatic, or migraine headache.

34. The method of claim 28, wherein the inflammatory disorder is osteoarthritis

35. The method of claim 28, wherein the inflammatory disorder is rheumatoid arthritis.

36. The method of claim 28, wherein the inflammatory disorder is migraine headache.

37. A method of treating or preventing a neurologic disorder in a subject comprising administering to a subject in need thereof a therapeutically effective amount of the composition of claim 28.

38. The method of claim 37, wherein the neurologic disorder is selected from the group consisting of Alzheimer's disease, dementia, Parkinson's disease, and migraine headache.

39. A method treating or preventing cancer in a subject comprising administering to a subject in need thereof a therapeutically effective amount of the composition of claim 28.

40. The method of claim 39, wherein the cancer is selected from the group consisting of colon cancer, pancreatic cancer, or breast cancer.
